(19) United States
(12) Patent Application Publication
(10) Pub. No.: US 2020/0386665 A1
(43) Pub. Date: Dec. 10, 2020

(54) SMART NANOPORE AND SOFT NANOPORE COMPOSITIONS FOR DETECTING AND UNFOLDING MISFOLDED PROTEINS AND METHODS OF USING SAME

(71) Applicants: THE TRUSTEES OF COLUMBIA UNIVERSITY IN THE CITY OF NEW YORK, New York, NY (US); UNIVERSITY OF VIENNA, Vienna (AT)

(72) Inventors: Angelo Cacciuto, New York, NY (US); Clarion Tung, Saratoga, CA (US); Ivan Coluzza, Gipuzkoa (ES)

(21) Appl. No.: 16/890,375

(22) Filed: Jun. 2, 2020

Related U.S. Application Data
(60) Provisional application No. 62/858,084, filed on Jun. 6, 2019.

(51) Int. Cl. G01N 15/10; G01N 33/487; G01N 15/12; B82Y 5/00

(52) U.S. Cl. CPC G01N 15/1056; G01N 33/48721; G01N 2015/0038; B82Y 5/00; G01N 15/12

(57) ABSTRACT

The present disclosure provides, inter alia, a device for capturing and unfolding a polymeric species (e.g., a misfolded protein) or disrupting aggregates of a polymeric species, the device including: a thin support and a plurality of nanopore structures piercing through the support, each nanopore structure having an inner surface and a void running the length of the structure, an outer boundary of the void being defined by the inner surface of the nanopore structure, the inner surface comprising hydrophobic regions capable of capturing and facilitating the unfolding of the misfolded polymeric species. Also provided are methods of separating and unfolding polymeric species, methods of treatment using these devices, and systems for measuring biomolecule transport, disaggregation and refolding in a liquid sample.

### CROSS REFERENCE TO RELATED APPLICATIONS

The present application is a U.S. Non-provisional Patent Application, which claims benefit of U.S. Provisional Patent Application Ser. No. 62/858,084, filed on Jun. 6, 2019. The entire content of the aforementioned application is incorporated by reference as if recited in full herein.

### GOVERNMENT FUNDING

This invention was made with government support under grant nos. DMR-1408259 and DMR-1703873 awarded by the National Science Foundation. The government has certain rights in the invention.

### FIELD OF DISCLOSURE

The present disclosure provides, inter alia, a device for capturing and unfolding a polymeric species (e.g., a misfolded protein) or disrupting aggregates of a polymeric species, and methods of using same in treating diseases or as research tools.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE DISCLOSURE

The three-dimensional conformation acquired by a protein in its functional form (native structure) is controlled by the sequence of amino acids along the protein backbone. The native structure is often unique for a given sequence. However, many catastrophic events can take place when just a few proteins fail to reach their functional configuration (Dobson et al. 1998). A significant obstacle along the correct folding pathway occurs when a protein aggregates with other copies of itself. The formation of large protein clusters can be lethal to cells and, in the long run, can lead to neurodegenerative diseases such as Alzheimer's and Parkinson's disease (Vendruscolo et al. 2001; Vendruscolo et al. 2009; Tartaglia et al. 2007; De Simone et al. 2012). Furthermore, protein aggregates present a significant obstacle in protein purification technology (Gräslund et al. 2008; Cheung et al. 2012). Since unregulated protein aggregation poses an important threat to life in all living organism, under evolutionary pressure, complex protection mechanisms against it have been set in place (Frydman et al. 2001; Baumketner et al. 2003; Kinjo et al. 2003; Jahn et al. 2008).

In prokaryotic cells, for instance, the GroEL/GroES chaperonin complex acts as an efficient protection against misfolding and aggregation. The GroEL/GroES chaperonin is a double-barreled complex with two large cavities where misfolded proteins are captured and isolated for a long time (˜15 s) and at considerable energy cost (7 ATPs per protein or 14 ATPs per cycle). The working principle of the GroEL/GroES has not been fully elucidated, but its primary function is to segregate misfolded proteins from the cytosol into a molecular cage to prevent their unregulated aggregation with other proteins. Furthermore, the GroEL is believed to help misfolded proteins captured in its interior to refold into their native state. Recently, a new refolding reaction pathway for the GroEL/GroES complex has been postulated (Coluzza et al. 2008; Coluzza et al. 2006). Coluzza et al. hypothesized that confinement inside the cage could induce protein translocation through the equatorial region that connects the two chambers, and suggested that the translocation process could help proteins escape local free energy minima regardless of their specific amino acid sequence. Although such a pathway has not been experimentally investigated, it offers an appealing strategy to promote the correct folding path of a protein and bypass their detrimental aggregation. The present disclosure is directed to this and other needs.

### SUMMARY OF THE DISCLOSURE

In the present disclosure, the translocation of a globular polymer was tested via a crude model for a misfolded globular protein, through a cylindrical pore whose inner surface is coated with a soft polymer brush. The complex interactions between the polymer, the brush, and the solvent were explored to understand under what conditions such a system could be useful as a device to refold misfolded proteins and/or break up the aggregates they form. The idea is to push the globular polymer through the pore using a flow field in the solvent and exploit the shear forces that develop from the interaction of the protein with the soft brush to break up agglomerates and unfold misfolded states. Crucially, a moving fluid in a pipe would itself generate shear forces due to the parabolic (Poiseuille) profile of the velocity field, even in the absence of the brush, and it has been shown in experiments and computer simulations, that large protein multimers like the von Willebrand factor (vWF) can unfold (Siedlecki et al. 1996; Schneider et al. 2007; Sing et al. 2010) as a result of the shear forces applied on the protein by a moving fluid. A recent review addresses the topic of shear-induced protein unfolding by comparing multiple experimental and theoretical studies on different proteins (Bekard et al. 2011). In most of these experimental setups, special flow devices are used to exert shear on the proteins in solution. Many of these experimental studies find an effect on the proteins function (or activity for enzymes) at moderate shear rates of 10²-10⁵ s⁻¹. However, the experiments in some of these devices include an air-water interface which can also contribute to a loss of protein functionality. In a different paper, Jaspe et al. investigated the behavior of a small protein in a channel of diameter equal to 180 μm. The fluid was pushed through the channel by a pressure drop leading to shear rates up to 10⁴ s⁻¹ (Jaspe et al. 2006). The authors found no sign of a significant structural change in the protein structure, and proposed a simple theoretical model to estimate the onset shear rate required to unfold their proteins. This is expected to be of the order of 10⁷ s⁻¹, which is very hard to achieve in small channels (Luo et al. 2017). The question of whether small proteins can unfold in physical shear flow remains controversial, whereas the induction of structural changes driven by fluid shear in larger complexes such as vWF is widely accepted (Bekard et al. 2011).

The present disclosure provides an explicit study of the unfolding pathway of a globular polymer driven by a fluid flow through a cylindrical pore coated with a deformable polymer brush. The goal is to understand under what conditions the presence of the brush can improve the refolding rate of the globular protein. Such an approach, combined with the scaling properties of the brush, offers the advantage that the setup can be scaled up to large pores that allow for fast flow velocities and a smaller likelihood of pore clogging by protein aggregates.

Although the equilibrium interactions between free chains with a cylindrical brush have been extensively characterized (Egorov et al. 2011), there has been no study investigating the ability of a brush to deform a globular polymer under flow. Of relevance to this work is also the study by Mahmood et al. who discussed the potential of a DNA-grafted cylindrical pore to function as a biosensor under the influence of an electrical field (Mahmood et al. 2014). Furthermore, studies on unfolding of polymer globules (Alexander-Katz et al. 2006), translocation under flow of star-polymers in a slit channel (Neratova et al. 2015) and rod-like proteins in both slit and cylindrical geometries (Posel et al. 2017) have also been recently published.

The present disclosure provides a “smart nanopore” than can detect and unfold misfolded polymeric species, such as, e.g., proteins. The working principle relies on the greater surface hydrophobicity of misfolded proteins. A properly folded native state protein packs its hydrophobic amino acids into its core, whereas a misfolded protein has many more hydrophobic amino acids on its surface. These exposed hydrophobic amino acids cause misfolded proteins to stick to one another and form aggregates.

By leveraging this principle, a smart nanopore with tuneable hydrophobic patterns on the pore interior is created. Proteins are injected into the nanopore and flow through. Native-state proteins avoid these patterned surfaces, but misfolded proteins and aggregates stick to these surfaces. In this way, the nanopore detects and captures misfolded proteins.

The unfolding is caused by a combination of the hydrophobic patterns and the solvent flow profile. The hydrophobic patterns cause the protein to spread, and the solvent flow profile puts a gentle shear on the protein. These two factors unfold the protein and pull it off the surface. Once unfolded, the protein is allowed a second chance to fold into the correct, native state.

The present disclosure also provides a “soft nanopore” that unfolds misfolded polymeric species, such as, e.g., proteins and polymeric, e.g., protein aggregates. Forced translocation through a cylindrical nanopore whose interior is decorated with a polymer “brush” can unfold misfolded proteins and aggregates.

Standard “hard” nanopores are smaller than the diameter of a protein, and as such, can be easily clogged, and may be unsuitable for high-throughput processes. The soft nanopore disclosed herein is much larger than a protein and so avoids this problem. Instead, the polymer brushes on the interior act to squeeze and unfold proteins and aggregates that pass through the soft nanopore. Once the proteins exit, they are given a second chance to fold into the correct structure.

Accordingly, one embodiment of the present disclosure is a device for capturing and unfolding a polymeric species or disrupting aggregates of a polymeric species, the device comprising: (a) a thin support; and (b) a plurality of nanopore structures piercing through the support, each nanopore structure having an inner surface and a void running the length of the structure, an outer boundary of the void being defined by the inner surface of the nanopore structure, the inner surface comprising hydrophobic regions capable of capturing and facilitating the unfolding of the misfolded polymeric species.

Another embodiment of the present disclosure is a method of separating an aggregate of polymeric species comprising the steps of: (a) contacting a solution comprising the aggregate with one side of a device disclosed herein; and (b) translocating the aggregate of the polymeric species through the nanopore structures of the device by applying a fluid force on the solution.

Another embodiment of the present disclosure is a method of unfolding a misfolded polymeric species comprising the steps of: (a) contacting a solution comprising the misfolded polymeric species with one side of a device disclosed herein; and (b) translocating the misfolded polymeric species through the nanopore structures of the device by applying a fluid force on the solution.

Another embodiment of the present disclosure is a method of separating a misfolded polymeric species from a mixture of correctly folded native species and misfolded species, the method comprising the steps of: (a) contacting the mixture with one side of a device disclosed herein; (b) applying a fluid force on the mixture sufficient to translocate the correctly folded native polymeric species through the nanopore structures of the device while the misfolded polymeric species become associated with the inner surface of the nanopore structures; and (c) collecting the properly folded polymeric species on the other side of the device.

Another embodiment of the present disclosure is a method of treating a subject suffering from a disease associated with aggregated protein molecules comprising the steps of: (a) obtaining sufficient amount of a body fluid comprising aggregated protein molecules from the subject; (b) contacting the body fluid with one side of a device disclosed herein; (c) passing the body fluid through the nanopore structures of the device by applying a fluid force on the body fluid to disrupt the aggregated protein molecules; (d) collecting the body fluid on the other side of the device; (e) repeating steps (b)-(d) as necessary; and (f) reintroducing the body fluid collected in step (e) into the subject so as thereby to treat the subject.

Another embodiment of the present disclosure is a method of treating a subject suffering from a disease associated with misfolded protein molecules comprising the steps of: (a) obtaining sufficient amount of a body fluid comprising misfolded protein molecules from the subject; (b) contacting the body fluid with one side of a device disclosed herein; (c) passing the body fluid through the nanopore structures of the device by applying a fluid force on the body fluid to unfold the misfolded protein molecules; (d) collecting the body fluid on the other side of the device; (e) repeating steps (b)-(d) as necessary; (f) allowing the unfolded protein molecules in the body fluid collected in step (e) to refold into the native conformation; and (g) reintroducing the body fluid from step (f) into the subject so as thereby to treat the subject.

Another embodiment of the present disclosure is a system for measuring biomolecule transport, disaggregation and refolding in a liquid sample, comprising: software programmed to run the system, and hardware that controls flow and pressure independently, wherein the hardware comprises the following devices connected in the following order: (a) a compressor that generates a pressure; (b) a pressure controller that controls the pressure generated by the compressor; (c) a filter; (d) a reservoir that holds the liquid sample; (e) a bubble trap and degasser; (f) a flow sensor that measures the flow rate of the sample; (g) an extruder in which a membrane with nanochannels is mounted; (h) a refractive index and/or fluorescence detector to analyze the liquid sample that flows through the membrane; and optionally (i) an automated collection unit to collect aliquots of the sample.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a schematic of a measurement setup according to the present disclosure. Arrangement of the setup: The pressure is generated by a compressor and is adjusted by a pressure controller (Elvesys OB1), after passing through a filter, the air or oil drives the liquid in the reservoir through the tubes. The reservoir remains degassed, because it's beneficial to use immiscible and incompressible oil instead of air to connect the pressure unit to the vial for long measurements. The liquid passes through a bubble trap and degasser (omitted from schematic) to reach the flow sensor, which measures the flow rate by a sinusoidal thermal heat pulse. The liquid next passes through an extruder in which the membrane with the nanochannels is mounted. A refractive index and/or fluorescence detector (HPLC-type setup) analyses the liquid that flows through the membrane. The samples are discarded or collected as volumetrically controlled aliquots.

FIGS. 2A-2C provides an example of a published method for making polymer-functionalized silicon nitride chips with nanochannel apertures in the range of ˜100 nm and channel lengths of ˜300 nm. The micrographs show chips modified with a polymer that allows the capture of protein inside the pores without restricting the flow through the channels.

FIG. 3 shows a commercially available anodized alumina membrane with 100-nm diameter pores used in the nanochannel flow setup.

FIG. 4 is a visualization of the relevant components of our system. The blue folded chain represents the protein model while the green chains are the polymers grafted to the cylinder walls forming the brush. The arrows indicated the direction of the fluid flow pushing the protein through the brush. The light blue dots indicate the SRD fluid particles.

FIG. 5 is a snapshot of a cylindrical brush under flow for Case 1 (see text for pore parameters), showing the side view on the left panel and the cross section on the right panel. Several chains are depicted in blue to show individual chain conformations. The solvent particles are not shown. The brush has chain length of Nₘ=10 and the fluid acceleration is a=0.1 and R=9.55σ.

FIG. 6 is a graph showing brush monomer density and solvent flow profile for a=0 and a=0.1 for the setup in case 1 and Nₘ=10.

FIG. 7 is a graph showing monomer density and solvent flow profiles for different chain lengths Nₘ for the setup in Case 1 with a=0.1. As the chain length Nₘ increases, the monomers fill the center of the core and the solvent velocity decreases.

FIG. 8 is a graph showing brush monomer density and solvent flow profile for a=0 and a=0.1 for the setup in Case 2 and Nₘ=20.

FIG. 9 is a graph showing monomer density and solvent flow profiles for different chain lengths Nₘ for the setup in Case 2 with a=0.1. As the chain length Nₘ increases, the monomers fill the center of the core and the solvent velocity decreases.

FIG. 10 is a graph showing brush monomer density and solvent flow profile for a=0 and a=0.1 for the setup in Case 3 and Nₘ=20. The top panel shows the result when averaging over the who extent of the pore, while the bottom panel shows the same data when only considering the polymer in a 106 shell the middle of the pore, i.e. for x=Lₚ/2.

FIG. 11 is a graph showing monomer density and solvent flow profiles for different chain lengths Nₘ for the setup in Case 3 with a=0.1. As the chain length Nₘ increases, the monomers fill the center of the core and the solvent velocity decreases.

FIG. 12 shows three snapshots from our simulations showing the translocation process of a globular polymer for the Case 1 pore, for a=0.1 and Nₘ=11. From top to bottom: the globular polymer pore entering the pore, the polymer in the middle of the pore, and the polymer exiting the pore. At the exit, additional shear forces arise due to the significant monomer density gradient.

FIG. 13 shows: (top row) the radius of gyration Rg of a globular polymer undergoing repeated translocation through the Case 1 pore, with flow a=0.1. The chain length Nₘ increases from left to right; and (bottom row) the corresponding position of the polymer, and at chain length Nₘ=12, the polymer cannot enter and the pore is essentially clogged.

FIG. 14 shows three snapshots from our simulations showing translocation of a globular polymer through the larger Case 3 pore with a=0.1 and chain length Nₘ=24. From top to bottom: the polymer enters the pore, the polymer in the middle of the pore, and the polymer just before exiting the pore.

FIG. 15 shows: (top row) the radius of gyration Rg of a globular polymer undergoing repeated translocation through the Case 3 pore, with flow a=0.1. The chain length Nₘ increases from left to right; and (bottom row) the corresponding position of the polymer. The larger pore allows for finer control of the monomer density gap via Nₘ, which in turn allows for better control of the polymer distortion during translocation.

FIG. 16 is a snapshot of a cylindrical brush under flow for Case 2 (see the Methods section of Example 2 for pore parameters), showing the side view on the left panel and the cross section on the right panel. Several chains are depicted in blue to show individual chain conformations. Here, Nₘ=20 and the ow acceleration a=0.1 and R=9.55σ.

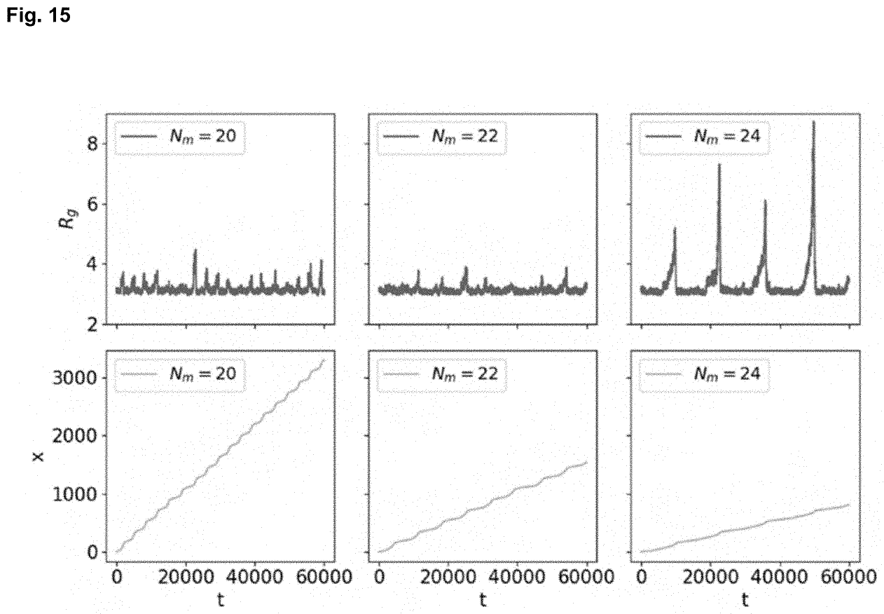

FIG. 17 is a snapshot of a cylindrical brush under flow for Case 3 (see the Methods section of Example 2 for pore parameters), showing the side view on the left panel and the cross section on the right panel. Several chains are depicted in blue to show individual chain conformations. Here, Nₘ=20 and the ow acceleration a=0.1 and R=19.1σ.

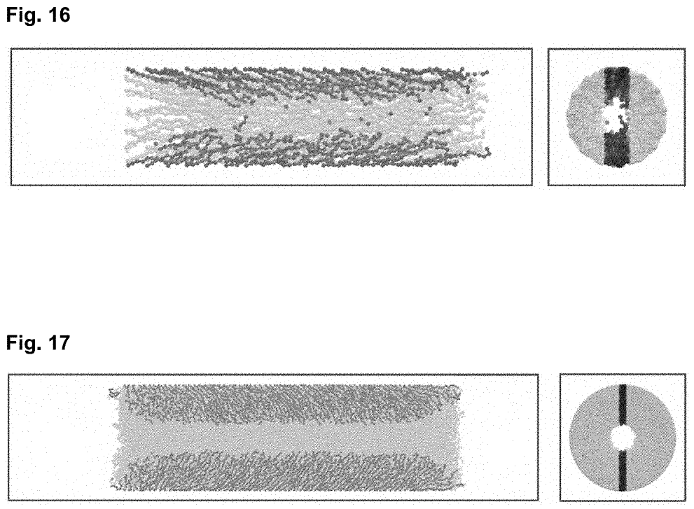

FIG. 18 shows in Case 1, the average tilt angle Φ of the chains in the brush with respect to the direction of the flow for different flow accelerations a. The top panel shows the results for Nₘ=10 and the bottom panel shows the results for Nₘ=14. The inset shows how for different values of a, the brush tilt Φ in the middle of the pore (x=Lₚ/2) is rather insensitive to the value of Nₘ. The lines are just guides for the eye.

FIG. 19 shows in Case 2, the average tilt angle Φ of the chains in the brush with respect to the direction of the flow for different flow accelerations a. The top panel corresponds to the case with Nₘ=30, the other to the case with Nₘ=40. The inset shows how Φ changes with a for different values of Nₘ in the middle of the pore (x=Lₚ/2). The lines are just guides for the eye.

FIG. 20 shows in Case 3, the average tilt angle Φ of the chains in the brush with respect to the direction of the flow for different flow accelerations a. The top panel corresponds to the case with Nₘ=20, the other to the case with Nₘ=24. The inset shows how Φ changes with a for different values of Nₘ in the middle of the pore (x=Lₚ/2). The lines are just guides for the eye.

FIG. 21 is a schematic representation of the prototype filters. In each pore of ˜150 nm in diameter a polymer brush is grown as depicted in the simulation snapshots (side view).

FIGS. 22A and 22B show two possible setups for the device. FIG. 22A shows bi-directional flow with a single porous surface in between. FIG. 22B shows unidirectional flow with multiple porous surfaces placed in series.

FIG. 23 is a graph of a first set of experimental data showing how the initial distribution of protein aggregates (in green) changes when passing through the filter (in red). The Blue curve corresponds to a full distribution containing monomeric proteins.

FIG. 24 is a graph of a second set of experimental data showing how the initial distribution of aggregated proteins (in green), changed after passing through the pore (red distribution). We believe the short peak at 2.2 nm is due to impurities, such as pNIPAM polymers that could have detached from the surface as a result of the fluid flow. The Blue curve corresponds to a full distribution containing only monomeric proteins. In this experiment the peak of the red curve seats where the expected folded distribution should be.

FIG. 25 shows the size histograms of initially aggregated (red) and native folded (yellow) BSA. The blue histogram shows the size distribution after one pass through a chip with ˜150 nm pores functionalized with dense polymer brushes at a concentration of 10 mg/m L.

FIG. 26 shows the critical shear found previously (g=0.01).

FIG. 27 is a structural diagram showing the average axial end-to-end distance R_{z} of the chain.

FIG. 28 shows axial end-to-end distances R_{z}/Nₐ for an N=50 polymer (g=0.008). At low wall hydrophobicity ε_{ω}, the polymer remains a globule. As ε increases, unfolding events are observed.

FIG. 29 shows three snapshots along a translocation trajectory of the Shea protein. Left: the metastable protein is outside the pore. Center: the protein begins to unfold as it is squeezed into the brush. Right: the unfolded protein exits the pore.

FIG. 30 shows the free energy landscape of the Shea protein. RMSDs along a translocation trajectory beginning in the misfolded state and refolding resulting in the native state are plotted, showing the unfolding-refolding mechanism. The color bar shows free energies in units of k_{B}T.

FIG. 31 shows the free energy landscape of the Coluzza protein. RMSDs along a translocation trajectory beginning in the misfolded state and refolding resulting in the native state are plotted, showing the unfolding-refolding mechanism. The color bar shows free energies in units of k_{B}T.

FIG. 32 shows histograms of the RMSDs of the Shea (top) and Coluzza (bottom) proteins before and after translocation, with polymer chain length 13 and force 8. Starting with ensembles of metastable proteins, forced translocation through the nanopore helps to unfold the proteins and give them a second chance to fold into the native state.

FIG. 33 is a structural diagram showing the unfolding rates for the Shea protein using nanopores with varying polymer chain lengths and driving forces. Simulations reveal a large region of parameter space in which unfolding can occur. Unfolding is defined as having a RMSD>2 upon exiting the nanopore. Circles denote successful unfolding, and their relative sizes scale with the unfolding efficiency. Squares denote translocation without unfolding, and x's denote no translocation.

FIG. 34 shows the radial monomer density profiles of the soft nanopore. Top: Increasing the chain length of the polymers (cl) shrinks the density gap at the pore center. Bottom: Stronger flow forces cause tilt the polymer chains, thereby increasing density gap. For cl=13 and f=10, the density profile has a gap whose width is smaller than the protein, and these parameters correspond to the highest unfolding rate.

FIG. 35 shows that the forced translocation through the soft nanopore can break apart protein aggregates Left: Dimer of two Shea proteins. Center: dimer entering the pore. Right: two separate proteins exit the pore

FIG. 36 shows a 9-mer of the Shea protein created by co-folding at high densities.

FIG. 37 shows that the 9-mer is broken into smaller pieces after exiting the soft nanopore.

FIG. 38A shows a top down view of a representative device according to the present disclosure.

FIG. 38B shows a partial side view of the device shown in FIG. 38A.

FIG. 38C shows a cross section view of the device depicted in FIG. 38A.

FIG. 38D shows a cross section view of a nanopore structure according to the present disclosure.

FIG. 38E shows a representative arrangement of multiple devices according to the present disclosure in series.

FIG. 39 shows a representative system according to the present disclosure.

### DETAILED DESCRIPTION OF THE DISCLOSURE

The present disclosure relates to a device capable of promoting the refolding of misfolded proteins and/or disassembly of protein aggregates. The device consists of an Aluminium-oxide surface about 10 microns in thickness and one square centimetre in area covered with nano-pores of diameter ranging from 100 nm to 200 nm piercing through it. These pores have an overall cylindrical shape and, along with the surface, are internally coated with a dense brush of Poly(N-isopropylacrylamide) pNIPAM polymers, which are flexible and water soluble. The device is immersed in water and partitions two regions of a small container. One side contains protein aggregates and/or misfolded proteins (e.g., left side), the other side is protein free. Using a pump or a simple syringe on the protein rich side of the container, the fluid can be set in motion, thus forcing the proteins and their aggregates to translocate through the pores before reaching the right side of the container.

It has been found that proteins and protein aggregates, when forced with a fluid flow to translocate through these pores, unfold and break apart, gaining a second chance at properly refolding. The coverage of the pores with the soft/deformable polymer brush is crucial in preventing the clogging of the pore by large protein aggregates. In fact, “hard” nanopores, that one could envision using for the same purpose, would need to have a diameter smaller than that of a protein to work, and as such, beyond the expense associated to the formation of such small pores, they are easily clogged, and unsuitable for high-throughput processes. The “soft” nanopores disclosed herein are much larger than a single protein and/or of the proteins aggregates so to avoid this problem. Indeed, it is the action of the polymer brushes the key element of the device as it acts to squeeze and unfold proteins and aggregates as they pass through the nanopore.

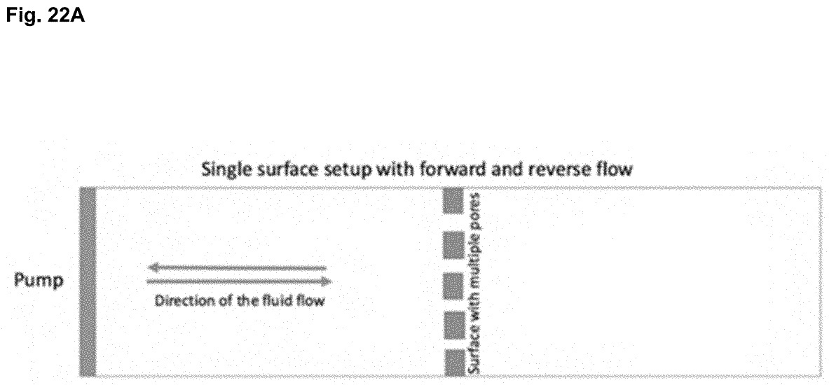

Using multiple surfaces in series or switching the direction of the flow against a single porous surface boosts the efficiency of device. See FIG. 22A and FIG. 22B for drawings of these two setups, and for more information on the experimental results.

It should be stressed that the specific material used for the surface that partitions the two regions of the container is not important, as long as the proteins do not stick to it. This is guarantee by the total coverage of the surface with the polymer. For instance, our results were obtained using Aluminum Oxide surfaces, however any porous surface, for instance a colloidal surface, once coated with the polymers will present an efficient barrier to the proteins. What is crucial for the functioning of the device is the size of the pores and the density of the brush within in. The specific choice of the polymer in the brush is also immaterial, as long as the polymers are soluble in water. For instance, the device will also work if the surface and the pores are coated with PEG (Polyethylene glycol) polymers rather than pNIPAM polymers. The pNIPAM is adopted because they are temperature responsive, and allow for a better control of the brush height coverage of the pore.

Numerical simulations were used to identify optimal brush densities to maximize the deformation and efficiency of the pore. Dynamic light scattering was used to experimentally test the functionality of the device. Bovine Serum Albumin (BSA) proteins were used herein. An initial sample at concentration of 10 mg/ml of BSA proteins denatured with temperature is forced through the device at a rate of 10 μL/min using a syringe. The data show that already after a single passage through the device the aggregates are efficiently broken bringing the new size population closer to the reference properly folded one.

Accordingly, one embodiment of the present disclosure is a device for capturing and unfolding a polymeric species or disrupting aggregates of a polymeric species, the device comprising: (a) a thin support; and (b) a plurality of nanopore structures piercing through the support, each nanopore structure having an inner surface and a void running the length of the structure, an outer boundary of the void being defined by the inner surface of the nanopore structure, the inner surface comprising hydrophobic regions capable of capturing and facilitating the unfolding of the misfolded polymeric species.

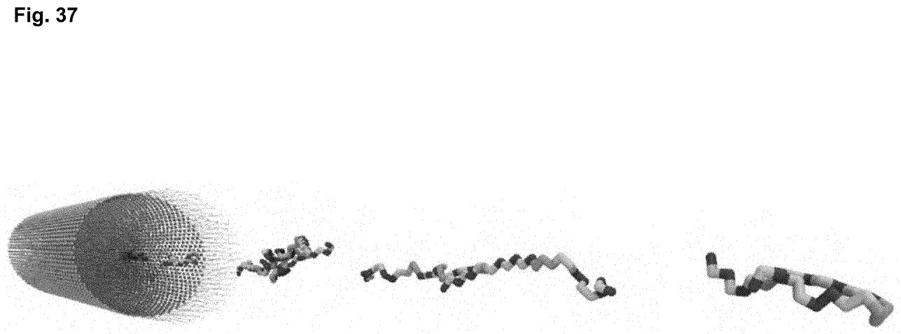

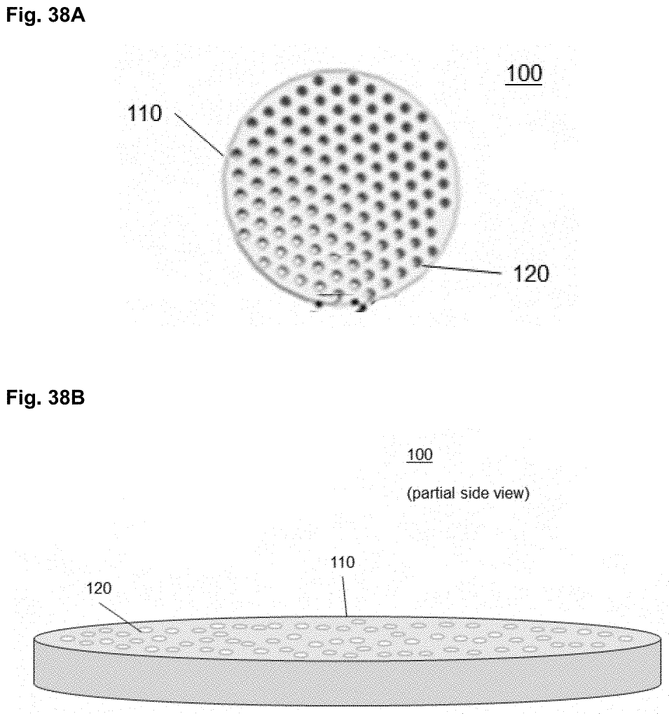

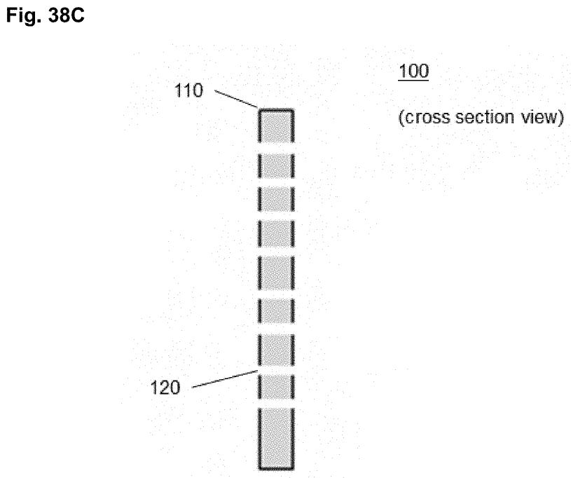

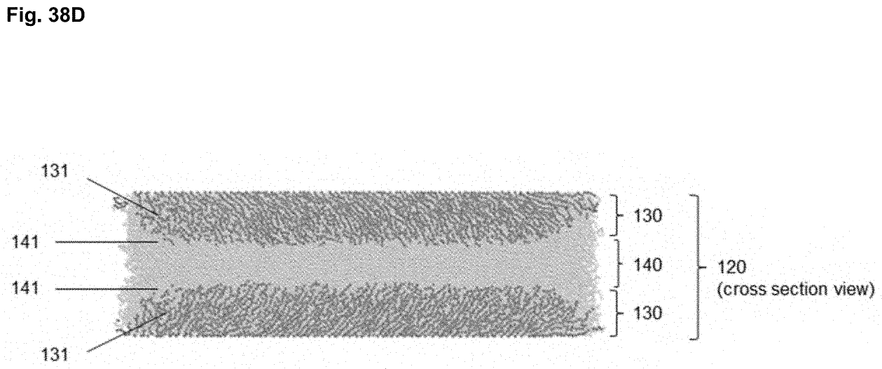

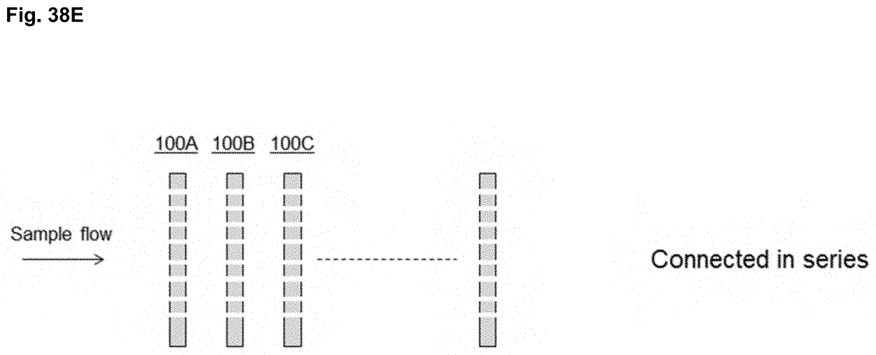

Turning now to FIGS. 38A, 38B and 38C, there is shown a representative device 100 according to the present disclosure (in top down (38A), partial side (38B) and cross section (38C) views). The device 100 includes a thin support 110 and a plurality of nanopore structures 120 piercing through the thin support 110. As shown in FIG. 38D, each nanopore structure 120 has an inner surface 130 and a void 140 running the length of the structure. An outer boundary 141 of the void 140 is defined by the inner surface 130 of the nanopore structure 120. The inner surface 130 comprises hydrophobic regions 131 that are capable of capturing and unfolding a misfolded polymeric species such as, e.g., a misfolded protein. As further shown in FIG. 38E, the device 100 may be operably connected in series with additional devices of the same construction (e.g., 100A, 100B, 100C . . . etc.).

In some embodiments, the support is of about 10 μm in thickness and about 1 cm² in area. In some embodiments, the support is made of a material to which the polymeric species do not stick, such as silica or aluminum-oxide.

In some embodiments, the nanopore structure has overall cylindrical shape with a diameter ranging from 100 nm to 200 nm.

In some embodiments, the polymeric species is a misfolded protein. As used herein a “misfolded protein” is a protein where the root mean squared deviation (RSMD) of the free energy, relative to the native conformation, has a value of greater than or equal to one, greater than or equal to two, greater than or equal to three, greater than or equal to four.

In some embodiments, the polymeric species is passed through the nanopore structures by a pressure driven flow of about 0.004 g to about 0.01 g.

As used herein a “hydrophobic region” is a non-polar region. In some embodiments, the inner surface of the nanopore device of the present disclosure has a hydrophobicity (ε_{w}) greater than or equal to about 1.0 k_{B}T, about 2.0 k_{B}T, about 3.0 k_{B}T, about 4.0 k_{B}T, about 5.0 k_{B}T, about 6.0 k_{B}T, about 7.0 k_{B}T, about 8.0 k_{B}T, or about 9.0 k_{B}T. In some embodiments, the inner surface of the nanopore structure has a hydrophobicity (ε_{w}) greater than or equal to 5.0 kBT.

In some embodiments, the hydrophobic regions comprise a plurality of polymer brushes. In some embodiments, the polymer brushes are made of polymers that is soluble in water. In some embodiments, the polymer brushes are made of polymers selected from PEG (Polyethylene glycol), PNIPAM (Poly(N-isopropylacrylamide)), or combinations thereof. In some embodiments, the polymer brushes have a chain length of 10 to 24 monomers. In some embodiments, the polymer brushes are capable of contacting the polymeric species, said contacting resulting in the disruption of the aggregates of the polymeric species or the unfolding of the polymeric species.

In some embodiments, a flow force is applied across the polymer brushes and creates a density gap at the center of the nanopore structure, and wherein the polymeric species is unfolded if the density gap is smaller than the size of the polymeric species. As used herein, a “density gap” is a region at the center of a nanopore structure wherein translocation of the polymeric species through the nanopore structure is relatively unobstructed by the polymer brushes; which region is defined by an outer boundary comprising the polymer brushes. In the present disclosure, an increased flow force will cause the polymer brushes to be pressed toward the inner surface of the nanopore structure, thus increasing the density gap, while increasing the chain length of the polymer brushes will decrease the density gap. In some embodiments of the disclosure, the density gap is between about 1 and about 8 amino acid residues wide. In some embodiments, the density gap is between about 2 and about 6 amino acid residues wide. In some embodiments, the density gap is about 4 amino acid residues wide.

In some embodiments, the nanopore structure has a radius of about 6 to about 20 amino acid residues. In some embodiments, the nanopore structure has a radius of about 6 to about 10 amino acid residues. In some embodiments, the nanopore structure has a radius of about 8 to about 9 amino acid residues. In some embodiments, the nanopore structure has a radius of about 8.5 amino acid residues. In some embodiments, the nanopore structure has a radius of about 8 to about 16 amino acid residues. In some embodiments, the nanopore structure has a radius of about 10 to about 14 amino acid residues. In some embodiments, the nanopore structure has a radius of about 12 amino acid residues.

Another embodiment of the present disclosure is a method of separating an aggregate of polymeric species comprising the steps of: (a) contacting a solution comprising the aggregate with one side of a device disclosed herein; and (b) translocating the aggregate of the polymeric species through the nanopore structures of the device by applying a fluid force on the solution.

As used herein, an “aggregate” of a polymeric species comprises two or more molecules of the species bound together. In some embodiments, the polymeric species in the aggregate are misfolded. In some embodiments, the aggregate is a protein aggregate.

As used herein, “translocate” and “translocating” mean movement of a polymeric species from one end of the void running the length of the nanopore structure to the other.

In some embodiments, the method further comprises the steps of: (c) once all the solution is on the other side of the device, repeating step (b) by applying a fluid force from the opposite direction; (d) repeating steps (b)-(c) as necessary; and (e) collecting the solution.

In some embodiments, the device is replaced with a plurality of same devices arranged in series.

Another embodiment of the present disclosure is a method of unfolding a misfolded polymeric species comprising the steps of: (a) contacting a solution comprising the misfolded polymeric species with one side of a device disclosed herein; and (b) translocating the misfolded polymeric species through the nanopore structures of the device by applying a fluid force on the solution.

In some embodiments, the misfolded polymeric species is a misfolded protein.

In some embodiments, the method further comprises the steps of: (c) once all the solution is on the other side of the device, repeating step (b) by applying a fluid force from the opposite direction; (d) repeating steps (b)-(c) as necessary; and (e) collecting the solution.

In some embodiments, the device is replaced with a plurality of same devices arranged in series.

In some embodiments, the method further comprises the step of: allowing the unfolded protein refold into its native conformation.

Another embodiment of the present disclosure is a method of separating a misfolded polymeric species from a mixture of correctly folded native species and misfolded species, the method comprising the steps of: (a) contacting the mixture with one side of a device disclosed herein; (b) applying a fluid force on the mixture sufficient to translocate the correctly folded native polymeric species through the nanopore structures of the device while the misfolded polymeric species become associated with the inner surface of the nanopore structures; and (c) collecting the properly folded polymeric species on the other side of the device.

In some embodiments, the misfolded polymeric species is a misfolded protein.

Another embodiment of the present disclosure is a method of treating a subject suffering from a disease associated with aggregated protein molecules comprising the steps of: (a) obtaining sufficient amount of a body fluid comprising aggregated protein molecules from the subject; (b) contacting the body fluid with one side of a device disclosed herein; (c) passing the body fluid through the nanopore structures of the device by applying a fluid force on the body fluid to disrupt the aggregated protein molecules; (d) collecting the body fluid on the other side of the device; (e) repeating steps (b)-(d) as necessary; and (f) reintroducing the body fluid collected in step (e) into the subject so as thereby to treat the subject.

In the present disclosure, diseases associated with aggregated protein molecules include, but are not limited to, Alzheimer's disease (AD), Parkinson's disease (PD), Huntington's disease (HD), amyotrophic lateral sclerosis (ALS) and prion diseases such as Creutzfeldt-Jakob Disease (CJD), Variant Creutzfeldt-Jakob Disease (vCJD), Gerstmann-Straussler-Scheinker Syndrome, Fatal Familial Insomnia, and Kuru.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, a “subject” is a mammal, preferably, a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. In some embodiments, the subject is a human.

As used herein, a “body fluid” is any fluid derived from a biological sample from the subject. In the present disclosure, biological samples include, but are not limited to, blood, plasma, cerebrospinal fluid, urine, skin, saliva, and biopsies. Biological samples are obtained from a subject by routine procedures and methods which are known in the art.

Another embodiment of the present disclosure is a method of treating a subject suffering from a disease associated with misfolded protein molecules comprising the steps of: (a) obtaining sufficient amount of a body fluid comprising misfolded protein molecules from the subject; (b) contacting the body fluid with one side of a device disclosed herein; (c) passing the body fluid through the nanopore structures of the device by applying a fluid force on the body fluid to unfold the misfolded protein molecules; (d) collecting the body fluid on the other side of the device; (e) repeating steps (b)-(d) as necessary; (f) allowing the unfolded protein molecules in the body fluid collected in step (e) to refold into the native conformation; and (g) reintroducing the body fluid from step (f) into the subject so as thereby to treat the subject.

Another embodiment of the present disclosure is a system for measuring biomolecule transport, disaggregation and refolding in a liquid sample, comprising: software programmed to run the system, and hardware that controls flow and pressure independently, wherein the hardware comprises the following devices connected in the following order: (a) a compressor that generates a pressure; (b) a pressure controller that controls the pressure generated by the compressor; (c) a filter; (d) a reservoir that holds the liquid sample; (e) a bubble trap and degasser; (f) a flow sensor that measures the flow rate of the sample; (g) an extruder in which a membrane with nanochannels is mounted; (h) a refractive index and/or fluorescence detector to analyze the liquid sample that flows through the membrane; and optionally (i) an automated collection unit to collect aliquots of the sample.

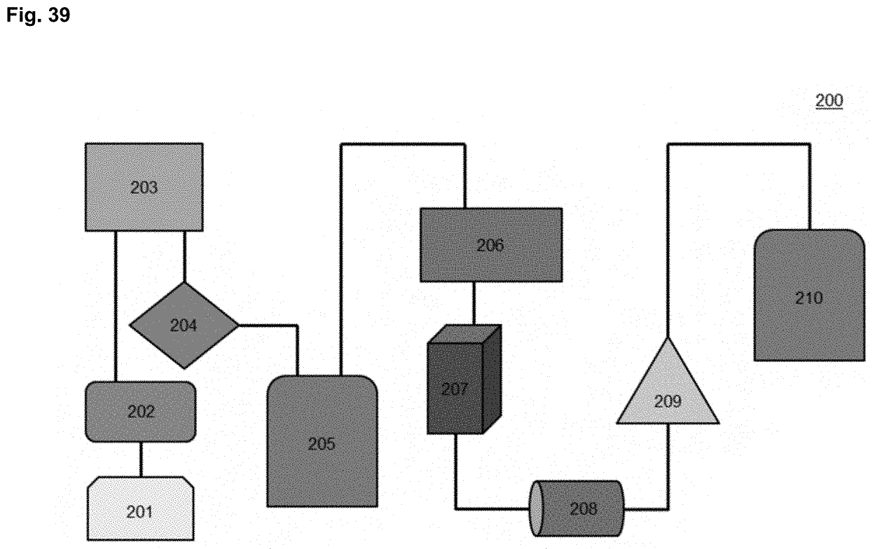

Turning now to FIG. 39, there is shown a representative system 200 according to the present disclosure in which software programmed to run the system 200 is housed, e.g., on a computer 201 that independently controls flow and pressure of the system 200. The system 200 further includes a compressor 202 that generates pressure to compress air or oil. The pressure is adjusted by a pressure controller 203. The compressed air or oil then passes a filter 204 and pushes a liquid sample held in a reservoir 205 through a bubble trap and degasser 206. The sample then sequentially passes a flow sensor 207 that measures the flow rate of the sample, an extruder 208 equipped with a membrane with nanochannels such as, e.g., the device 100 as described above, and a refractive index and/or fluorescence detector 209 that analyzes the liquid sample that flows through the membrane. The system 200 may optionally include an automated collection unit 210 to collect aliquots of the sample. The components and arrangement of such components as depicted in system 200 is exemplary only and may be arranged and/or modified to achieve the desired results as described herein.

It is noted that, in some embodiments, the hardware order may be varied so long as the goal of the system, e.g., measuring biomolecule transport and separation in a liquid sample, is achieved. Moreover, in some embodiments, the identified devices may be omitted or substituted with other conventionally known devices that accomplish substantially the same function. Furthermore, in some embodiments, each hardware device in the system may be used as a single device or multiple devices of the same function may be included.

In some embodiments, the hardware and software components of the present system may also be configured to process, store and communicate information and may include one or more computer systems, data storage systems and networking systems.

In some embodiments, the membrane is silicon nitride membrane, and in some embodiments, the membrane is anodized alumina membrane.

In some embodiments, the nanochannels have a length ranging from about 300 nm to about 100 μm, and have tunable apertures.

In some embodiments, the membrane with nanochannels is modified with dense polymer brushes. And in some embodiments, the polymer is poly(N-isopropyl acrylamide) (PNIPAM).

The following examples are provided to further illustrate certain aspects of the present disclosure. These examples are illustrative only and are not intended to limit the scope of the disclosure in any way.

### EXAMPLES

### Example 1

### Measuring Biomolecule Transport, Disaggregation and Refolding in a Liquid Sample

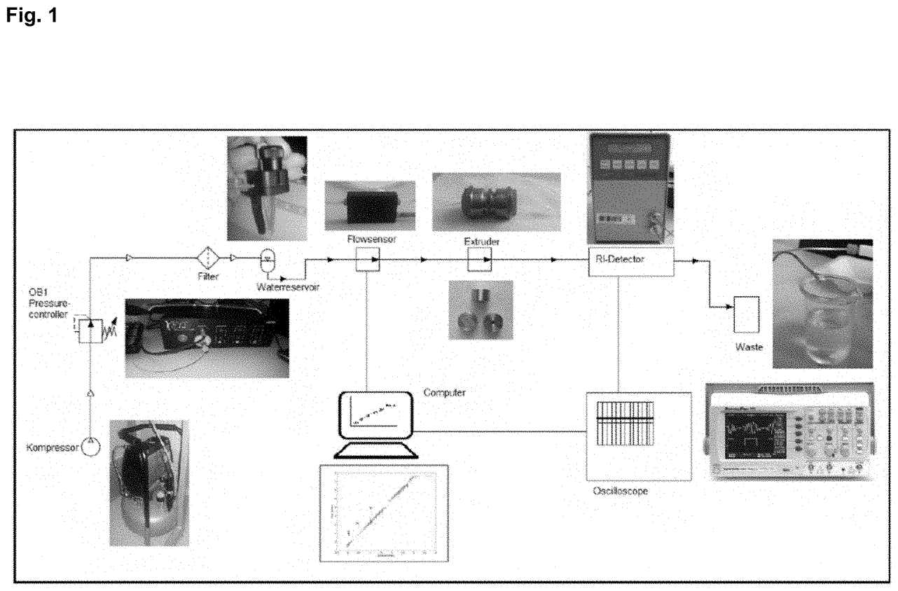

A rough schematic of the measurement setup is shown in FIG. 1. Software with a GUI that is able to control and record the flow and pressure patterns together with molecular transport via real-time optical detection is programmed and implemented to run the setup. This measurement system is enabled by hardware that can control and record the flow and pressure independently so as to avoid the usual assumptions or the calculation of pressure differences based on flow rates and channel dimensions. The calculation of pressure differences is required as the polymer-grafted nanochannels break new ground in terms of liquid and colloidal flow through confined spaces, for which models still require experimental verification. This setup can be combined with an automated collection unit to collect aliquots of e.g. protein samples for further analysis.

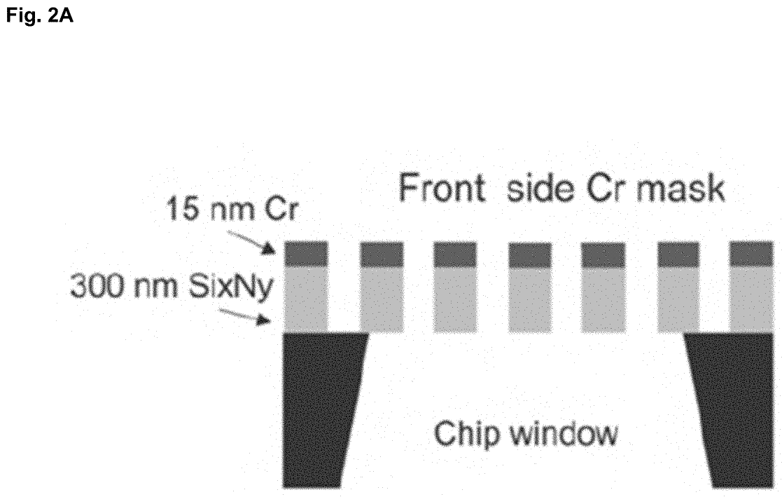

The nanochannels to be investigated are fitted into a custom-made extruder module and may be manufactured in different ways depending on the targeted application. The original versions featured short (˜300 nm) and well-spaced channels with tunable apertures in silicon nitride membranes, fabricated by cleanroom and colloidal nano- and microlithographic techniques (see FIGS. 2A-2C).

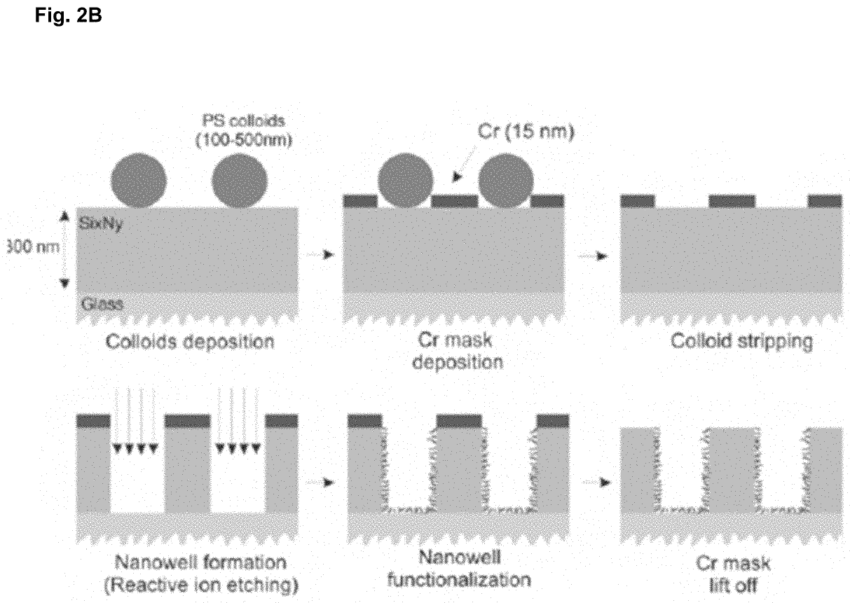

A more economical chip production method that features densely spaced and long (˜100 μm) channels employs anodized alumina membranes (see FIG. 3). Such membranes are commercially available; however, the ranges of aperture diameters, channel lengths and channel spacings are restricted.

The free-standing membranes patterned with nanopores may be modified with dense polymer brushes for most investigations of the properties and applications of polymer-functionalized nanochannels. The chips are first modified with an initiator for radical polymerization via gas-phase silanization, which creates a dense initiator coverage on both types of membranes, including in the channels. By controlling the initiator density, the desirable polymer brush grafting density is achieved, which is a major determinant of brush properties. Controlled polymerization that creates a uniform brush with a defined molecular weight (degree of polymerization) of the polymer chains grafted to the pore wall is achieved either ex-situ or in-situ. In this step, it is important to ensure the continuous supply of monomer for polymerization also in the restricted volume of the nanopores, and thus a setup for polymerization that ensures an exchange of the monomer-containing volume within the nanopores either intermittently or continuously during polymerization is required. A suitable polymer for most investigations is poly(N-isopropyl acrylamide) (PNIPAM), which is a hydrophilic polymer that can be grown by atom transfer radical polymerization (ATRP) from initiator. It forms a hydrophilic brush that repels protein adsorption at room temperature, but can be collapsed to create a hydrophobic thin polymer coating around body temperature. The hydrated polymer brush thickness is varied by the polymerization time, but typically a thickness is selected based on the pore radius that the brush thickness is expected to correspond to. This reduces but does not inhibit the flow of liquid through the nanopores and is able to control the transport of macromolecules such as proteins through the nanoporous membrane via the flow conditions. A large number of pores in parallel (cf. sample dimensions in FIG. 3) are analyzed for all chips to allow for measurements in a range where flow rates can be accurately produced and measured, and to provide sufficient amount of sample for analysis on the trans-side of the polymer-grafted membrane.

### Example 2

### Numerical Simulations of a Globular Polymer Translocation

### Methods

Throughout the present disclosure, the data are represented in dimensionless Lennard-Jones units, for which the fundamental quantities mass m₀, length σ₀, epsilon ε₀, and the Boltzmann constant k_{B} are set to 1, and all of the specified masses, distances, and energies are multiples of these fundamental values corresponding to T=T₀=ε₀/kB, m=m₀, σ=σ₀, and

$${τ}_{0} = \sqrt{\frac{{m}_{0} {σ}_{0}^{2}}{{ϵ}_{0}}} .$$

Each polymer grafted on the inner surface of the pore of radius R is described as a sequence of spherical beads of diameter a. Excluded volume interactions between any two monomers are enforced via a Weeks-Chandler-Andersen (WCA) potential

$${U}_{WCA} = 4 ϵ [ {( \frac{σ}{r} )}^{12} - {( \frac{σ}{r} )}^{6} + \frac{1}{4} ] \qquad ( 1 )$$

extending up to

$${r}_{c} = {2}^{\frac{1}{6}} σ$$

with ε=k_{B}T. Connected monomers along the chain are held together with a FENE potential of the form

$${U}_{FENE} = \frac{{KR}_{0}^{2}}{2} ln [ 1 - {( \frac{r}{{R}_{0}} )}^{2} ] \qquad ( 2 )$$

Where R₀=1.5σ is the maximum bond length and K=30k_{B}T/σ² is the strength of the bond. The surface of the cylinder is covered with densely packed WCA spherical particles of diameter a arranged according to a hexagonal lattice with lattice constant equal to a. These particles are locked in place during the simulation, and each polymer has its first monomer linked to one of them with the same FENE potential described above. The pore extends along the x axis of our simulation box up to a length Lₚ and contains Np polymers of length Nₘ, at a grafting density ρ_{G}=Nₚ/(πR²Lₚ).

The polymer that translocates through this pore is described in a similar manner, with the exception that each of its 200 monomers of diameter a is connected to its neighbor with harmonic bonds with a minimum at a and spring constant K₀=200k_{B}T. The strong spring constant is equivalent to a constant bond length equal to a as in the protein models by Honeycutt and Thirumalai (Honeycutt and Thirumalai, 1992). The monomers interact with a Lennard-Jones potential of the form:

$${U}_{LJ} = 4 ϵ [ {( \frac{σ}{r} )}^{12} - {( \frac{σ}{r} )}^{6} ] \qquad ( 3 )$$

The cutoff is set to 2.5σ and e=k_{B}T, which yields a globular polymer at equilibrium. The solvent is described by multiparticle collision dynamics, also known as stochastic rotation dynamics (SRD), a particle-based mesoscopic method used to reproduce hydrodynamic flow fields and solute interactions. The method consists of two steps. In the streaming step, particles move according to rᵢ(t+Δt_{SRD})=rᵢ+vᵢΔt_{SRD}. In the collision step, SRD particles are assigned to cubic bins of length Δx, the center of mass velocity v_{c m} is calculated, and the relative velocities are rotated by an angle a about a random axis, according to vᵢ(t+Δt_{SRD})=v_{c m, i}(t)+Ω(α)(vᵢ(t)−v_{cm, i}(t)), where is a rotation angle. We set the SRD particle mass m_{SRD}=0.1m, the average particles per bin ρ=10, the bin size Δx=σ, the SRD timestep Δt_{SRD}=0.01τ and rotation angle α=120°, giving a fluid viscosity of η=7.55. All monomer masses are set to m_{M}=ρm_{SRD} and are coupled to SRD particles in the collision step. Solvent flow is induced by applying a constant acceleration a to all solvent particles, and all subsequent values are reported in units of σ/τ². SRD particles are confined within hard cylindrical walls with the same axis and radius R as the pore, and length L spanning the length of the simulation box with periodic boundary conditions. To accurately represent no-slip boundary conditions at the walls of the cylinder, we use the bounce-back and bulk-filling rules described by Lamura (Lamura et al. 2001).

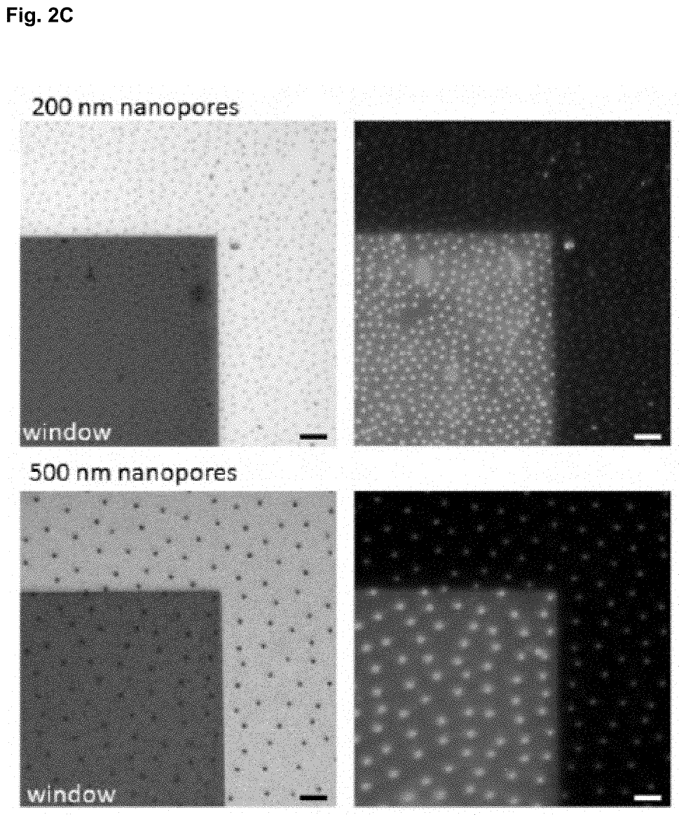

FIG. 4 shows a typical snapshot of the system including all components considered in this study, i.e. the cylindrical channel, the pore, the brush and the globular polymer.

Finally, the repulsion between any monomer and the walls of the cylinder is described using a WCA potential of the form

$${U}_{Wall} = 4 ϵ [ {( \frac{σ}{( {R}_{i} ( y , z ) - R )} )}^{12} - {( \frac{σ}{( {R}_{i} ( y , z ) - R )} )}^{6} + \frac{1}{4} ] , \qquad ( 4 )$$

with ε=k_{B}T and cutoff

$${2}^{\frac{1}{6}}$$

σ. Here (Rₜ(y, z)−R) is the radial distance of monomer i from the surface of the cylinder, and R is the cylinder radius. In this work we considered two pore sizes, one of radius R=9.55σ and the other R=19.1σ. Before the globular polymer is translocated through the pore, the brush is equilibrated in the presence of the fluid flow.

The simulations are performed using a timestep of Δt=0.002τ and simulations are run for a minimum of 10⁸ timesteps.

Given the large number of parameters associated with this system, and the lengthy nature of the simulations with an effective, yet explicit fluid, we are limited to study a subset of possible parameters. We considered three explicitly brush setups separately. The first case, Case 1, is characterized by a pore of radius R=9.55σ, length Lₚ=71.7σ, and grafting density ρ_{G}=0.28. In this case we considered brush polymer chains with Nₘ=10, 11, 12, 14 monomers, as larger values of Nₘ would overfill the pore. The second case, Case 2, is characterized by a pore with the same radius and length as in Case 1, but with a smaller grafting density ρ_{G}=0.07. Here, we considered brush polymers consisting of Nₘ=20, 30, 40 and 50 monomers. Finally, the third case, Case 3, is characterized by a pore of R=19.16, Lₚ=103.16 and ρ_{G}=0.28, a setup essentially equivalent to Case 1 with a pore diameter twice as large. For this case, we considered brush polymers with Nₘ=20, 22, 24 and 26 monomers. To study the crucial finite size effects introduced by the boundaries of the pore we considered simulation boxes of lengths L>Lₚ. For Case 1 we selected L=92.7σ, for Case 2 we selected L=112.7σ, and for Case 3, we set L=183.1σ. For all cases, we considered fluid accelerations in the range a E [0.0, 0.1], corresponding to Reynold's numbers ranging from Re∈[0.0, 0.05]. As a reference, if we consider a large multimeric protein like the vWF-factor, and set a=80 nm, that would give a pressure drop

$$\frac{Δ p}{Δ L} = 0.9$$

bar/mm for a=0.1.

### Characterization of the Pore

The analysis is started by characterizing the behavior of the brush under the influence of the fluid flow and in the absence of the globular polymer. This is important because, intuitively, one would expect that when the brush is long enough to fill the pore, a translocation event will force a globular polymer to deform as it squeezes through it. In the opposite case, when the brush profile allows for a monomer-free region at the center of the pore much larger than the radius of gyration of the globular polymer, the globule can translocate through the pore with minimal disturbance from the brush. An optimal brush will have a monomer density gap along the pore axis whose size is comparable or smaller than the size of the protein.

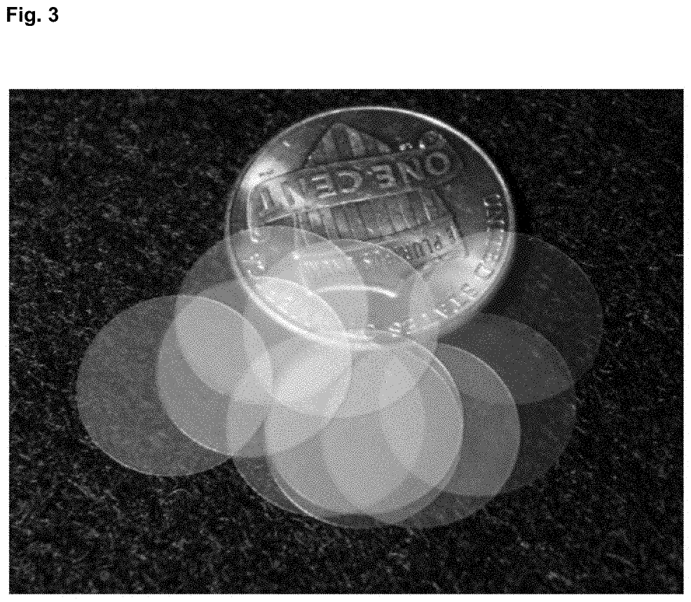

In Case 1, FIG. 5 shows a typical steady state configuration of the brush under a flow with a=0.1. The side-view and the cross-section are shown independently, and a small monomer density gap can be observed at the pore center.

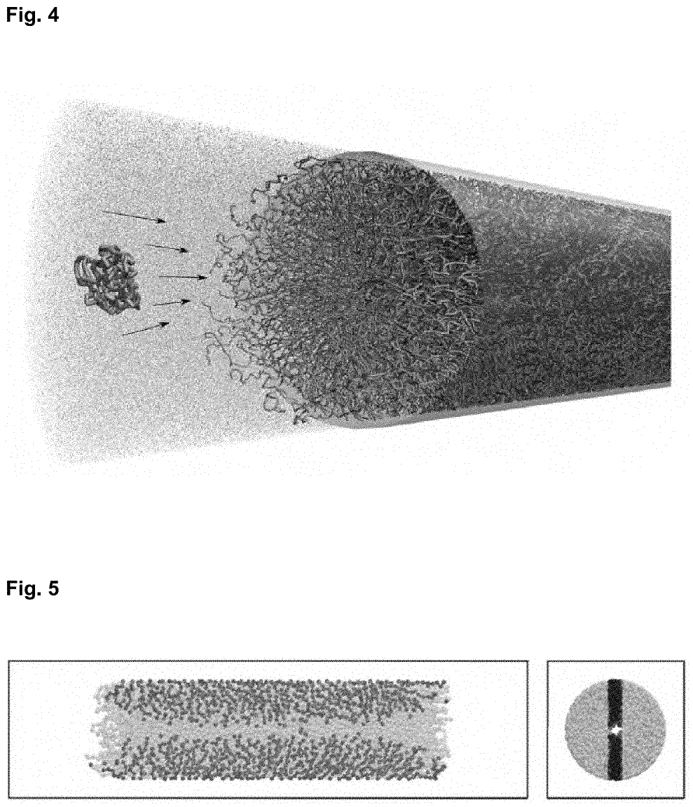

FIG. 6 shows both the radial profile of the axial velocity of the fluid and the monomer density profile for a=0 and a=0.1. The density profile under flow is almost identical to the one at equilibrium, and it is characterized by a density gap at the pore center and increasing monomer density approaching the wall. Significant layering effects are seen near the wall due to the high grafting density. The solvent flow profile for a=0.1 shows a non-insignificant fluid velocity along the axis of the pore, followed by a long plateau that persists deep into the brush until it finally drops to zero at the cylinder wall. To properly handle the no-slip boundary condition at the cylinder wall, we modified the default SRD implementation in LAMMPS (Plimpton, 1995) to include the corrections discussed in Lamura et al. (Lamura et al. 2001). Simulations of the fluid flow inside the cylinder when no polymer brush is included show an insignificant slip of the fluid near the wall. It is not clear to what extent the long and weak plateau observed for r>4 is an artifact of the specific coupling of the SRD particles and the monomers, or this can be understood, even at such a small length scales, through the frame of Darcy's law describing a fluid flow in a porous medium. Either way, this effect is rather weak and should have no significant impact on our results on the translocation of the globular polymer through the pore.

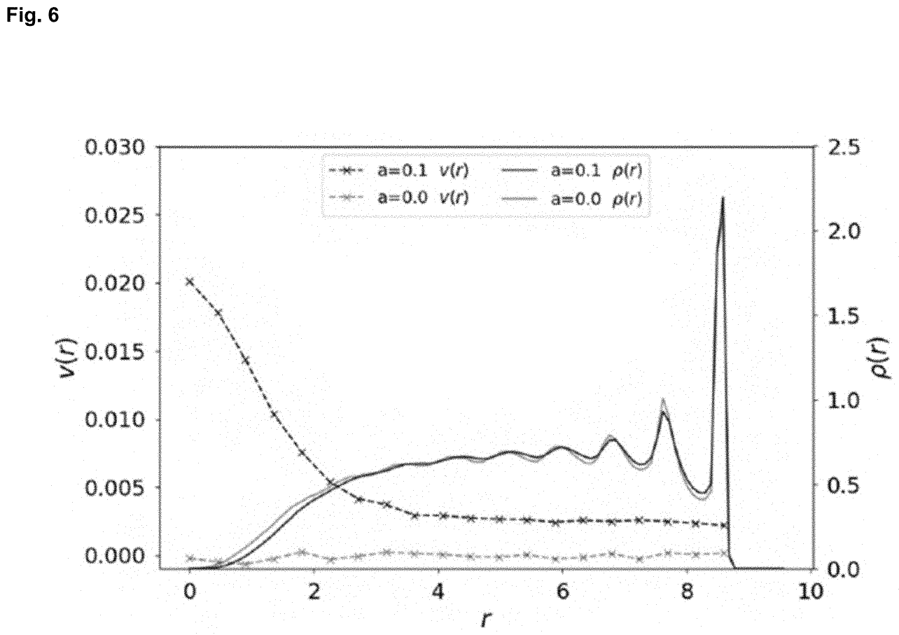

We now investigate how increasing chain length Nₘ changes the monomer density and solvent velocity profiles at a=0.1. Results of this analysis are shown in FIG. 7.

The main change in the density and velocity profiles occurs at the center of the pore. As Nₘ increases, the monomers gradually and systematically fill the density gap at the pore center. As expected, the decrease of the size of the density gap at the pore center is followed by a significant drop in the flow velocity profile. These data indicate that when the brush polymer chains are too long the pore becomes effectively clogged against the solvent flow, thus making for a poor candidate as a device for refolding translocating globular polymers. In the opposite limit, when the brush is too short, no significant interactions between the brush and a translocating globule can be established due to a density gap that would be larger than the radius of gyration of the globule. We identify Nₘ≈10 as an optimal candidate under these solvent flow conditions, as there is still a sizable density gap at the pore center that allows for solvent flow.

Snapshots of the brushes show that along the pore's cylindrical axis, the monomer density is mostly uniform in the middle but varies greatly at the edges of the pore, due to the splay of the individual polymers exiting the pore. A detailed analysis of the orientation Φ of the brush with respect to the flow velocity is presented in the Supplementary Material section below. In summary, the brush acquires a symmetric shape at the center of the pore (x=Lₚ/2) at equilibrium (a=0), with Φ 90 in the middle, Φ>90 for x<Lₚ/2, and Φ<90 for x>L_{I}/2. As the flow acceleration a increases, the average value of 0 across the pore becomes smaller, indicating that the brush begins to tilt towards the direction of the flow. Interestingly, the polymers at the pore entrance (x≅0) point against the flow Φ>90 even for large flow values of a (the larger Nm the stronger this effect), while at the pore exit (x Lₚ) the polymers are well stretched in the direction of the flow (Φ≅20°). We also find the degree of tilting to be rather insensitive to different chain lengths Nm near the middle of the brush, and that the density profile of the brush computed by only considering polymers the middle of the brush is independent of the fluid acceleration a (data not shown). This suggests that at this grafting density the tilting polymers must compensate for the unchanged brush height by stretching.

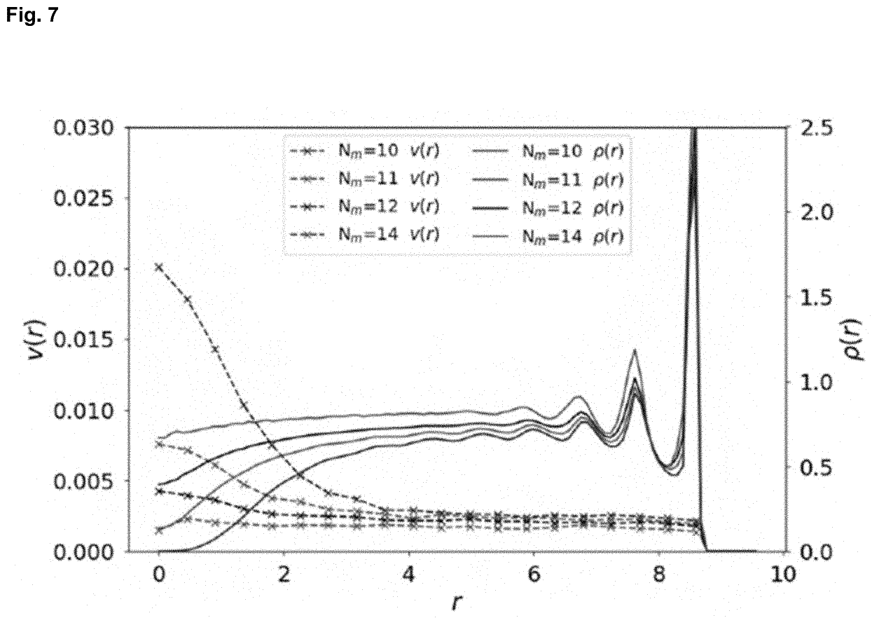

Now consider the second pore considered in this study: Case 2 with lower grafting density. The lower grafting density makes the brush more deformable under solvent flow. Here, the brush acquires large tilt angles for large flows and it compresses against the wall. The monomer density profiles both with and without solvent flow are shown in FIG. 8. At a=0, monomers fill the pore center, but at a=0.1, a large density gap develops in the center of the pore and the monomer density near the walls increases as a result of the brush compression against the walls of the cylinder. The solvent velocity profile shows a parabolic functional form at the pore center.

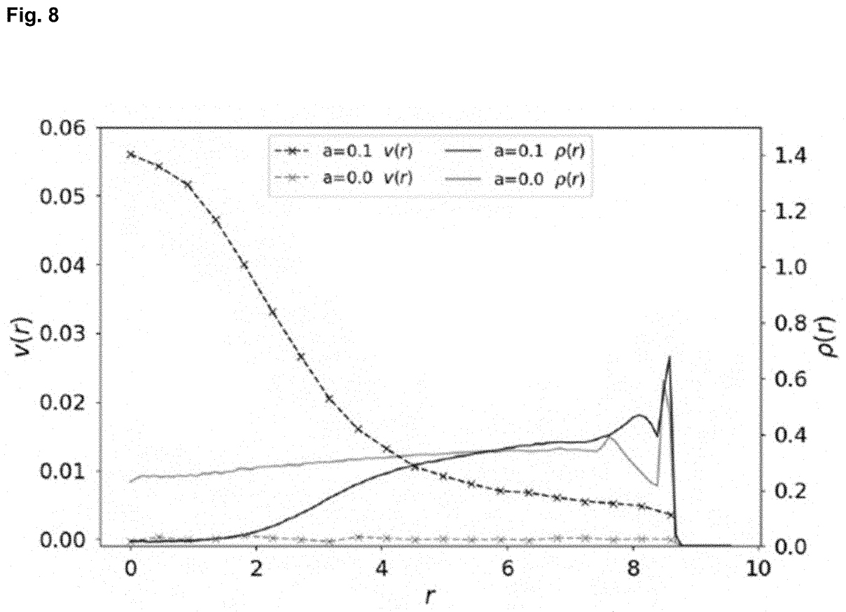

FIG. 9 shows how the density and velocity profiles change upon increasing the length of the polymers Nₘ. Crucially, when comparing Case 1 with Nₘ=10 and Case 2 with Nₘ=40, two brushes with the same overall monomers density inside the cylinder, we find that in the system with the lower grafting density (Case 2) the pore is completely occluded by the monomers, whereas the pore with the higher grafting density (case 1) shows a sizable monomer density gap at its core. This indicates that the grafting density plays a crucial role on the overall conformation of the brush under flow. To better characterize the brush conformation in the presence of the fluid flow, we also measure the brush tilt angle Φ as before. A study of the brush tilt angle as a function of the flow for different polymer lengths shows that (see Supplementary Material section below for details) at this low grafting density, the brush tilts much more sharply than in Case 1, and already for a≥0.05, the brush is already fully stretched throughout the length of the pore. Furthermore, as was the case for the pore with higher grafting density, the tilt angle, in the middle of the pore, (x≅Lₚ/2), seems to be rather insensitive to the chain length Nₘ.

Given the strong dependence of the density profile on the fluid flow for Case 2, it is more difficult to control the size of the monomer density gap, which is a critical design parameter for a refolding device. Furthermore, the configuration of the brush at the target acceleration a=0.1 consists of stretched polymers aligned along the direction of the flow throughout the pore, forming what is basically a soft funnel that is unlikely to generate sufficiently large shear forces on a translocating globular polymer.

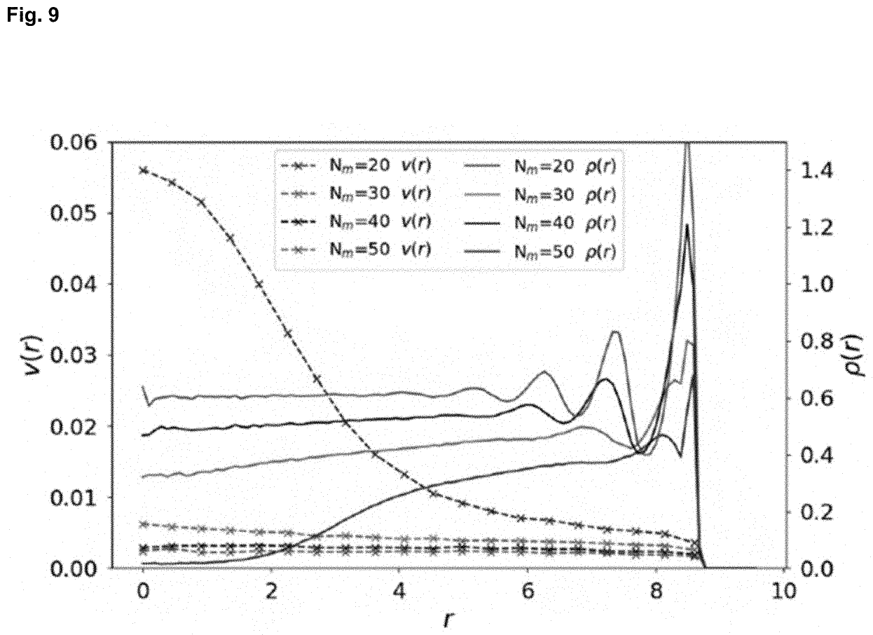

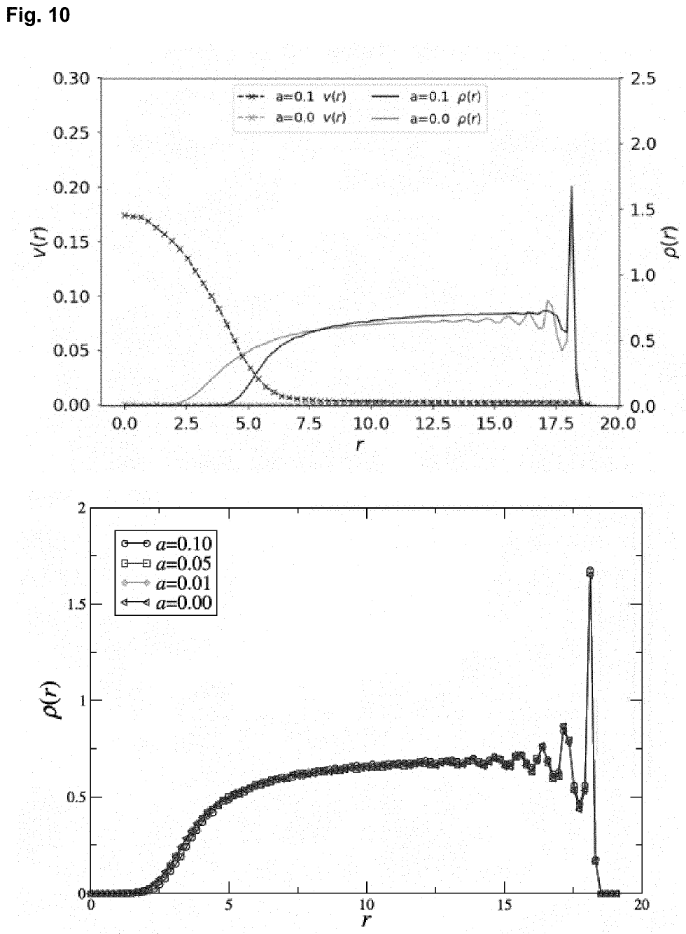

We now turn our attention to Case 3 which is a pore with twice the radius but the same grafting density as the pore in Case 1. We emphasize that we keep the same maximum applied fluid accelerations a=0.1 used in the other two cases. This clearly results in a larger fluid velocity inside the pore as expected from Poiseuille's law. We made this choice because we want to investigate how a change of the channel radius would affect velocity and density profiles inside the pore while keeping the fluid driving force constant. Because of the faster fluid inside the pore, overall, we expect that the brush shows a more substantial tilt than in Case 1, but not as dramatic as it is in the brush at lower grafting density in Case 2. FIG. 10 (top) compares monomer density and solvent velocity profiles for the driven (a>0) and the equilibrium systems (a=0). In the presence of a fluid flow, the monomer density near the walls is similar to that at equilibrium, albeit with weaker layering, and the size of the monomer density gap at the center of the pore is somewhat wider than that at equilibrium. This result would suggest that unlike the behavior of the pore in Case 1, not only the chain conformation, but also the size of the monomer density gap depend on a. A more careful analysis that only considers the monomer density profile around the middle of the pore (x≅Lₚ/2), which should minimize the effect of the larger edges at the pore opening and exit, reveals that this is not the case, and as observed in Case 1 with the smaller pore radius, the monomer density distribution q(r) is independent of a. The result of this analysis is shown in FIG. 10 (bottom). The effect on the density profile due to increasing the chain length Nm is shown in FIG. 11. The data indicate a systematic filling of density gap followed by a decrease of the flow velocity within it as Nm increases. A detailed study of the tilt angle of the brush along the pore for different values of a is presented in the Supplementary Material section below. Here, we summarize the results by mentioning that, as in Case 1, the front of the pore is characterized by a layer of polymers that resist pointing in the direction of the flow even at the highest driving forces (this effect becomes more evident as one increases Nₘ) while the back side of the brush adapts to the flow. We also observe, as was the case in the previous two cases, that while Φ decreases with fluid acceleration, it remains basically independent of the number of monomers in the polymer brush, Nₘ, for a fixed value of a.

It is important to stress that since the statistical properties of the pores coated with the polymer brush at the highest grafting densities are independent of the presence of the fluid flow, at least when it comes to the density profile and the size of the monomer-free gap along the pore axis, it should be possible to systematically scale up the pore diameter, as we did going from Case 1 to Case 3, and use standard equilibrium arguments to estimate the expected brush height in the pore (Alexander, 1977) even when in the presence of the fluid flow. This is important because larger pores that have the advantage of generating larger flow velocities in their cores at a fixed driving force, are less likely to be clogged by protein aggregates, and are easier to fabricate. Furthermore, as discussed by Dimitrov et al. (Dimitrov et al. 2006) (and references therein), a convenient property of cylindrical polymer brushes is that as the tube diameter increases, the reduced brush height h/Nₘ decreases. This allows for a finer control of the brush height with the chain length Nₘ, and thus a better control of the monomer density gap at the pore center.

It is worth noting that several studies on polymer brushes under shear have been published and, consistently with our results (at least in the large grafting density regime), the brush height is expected to be rather insensitive to the applied shear rate (Yarin, 1990; Binder, 2002; Rabin and Alexander, 1990). It should be stressed, however, that most of these works were performed on planar brushes of infinite extension. In our system, we considered finite-sized pores, and in our data, as discussed above, edge effects can become important when studying the statistical properties of the brush. Although we expect the data collected in the middle of the brush at x≅Lₚ/2 to be rather insensitive to the boundaries, for a systematic study of the scaling laws of a cylindrical polymer brush under shear, one should ideally consider a setup where the side length of the simulation box equals the length of the pore (L=Lₚ) with periodic boundary conditions to mimic the behavior of an infinitely long pore. This is not that study, because as it turns out, the effect of the edges is crucial when considering the translocation of the globular polymer through the pore. In fact, the largest shear forces develop at the pore exit as the globular polymer moves from a high monomer density region to a depleted one.

### Translocation Events

We identify the brush with Nₘ≅10 in Case 1 and the brush with Nₘ≅24 in Case 3 as the most promising re-folders. In both cases the brush grafting density is sufficiently large that the monomer density profiles in the middle of the pore are not affected by the solvent flow, and a sufficiently wide monomer density gap is available to interact with the globular polymer without dramatically reducing the velocity of the flow through the pore. As the globular polymer translocates through the pore, we characterize its degree of distortion using its radius of gyration R_{g}. As a reference the radius of gyration of our globular polymer with 200 monomers at equilibrium is measured to be R_{g}=3.09σ. The polymer theta point was measure to be at roughly T_{θ}=2 (Parsons and Williams, 2006).

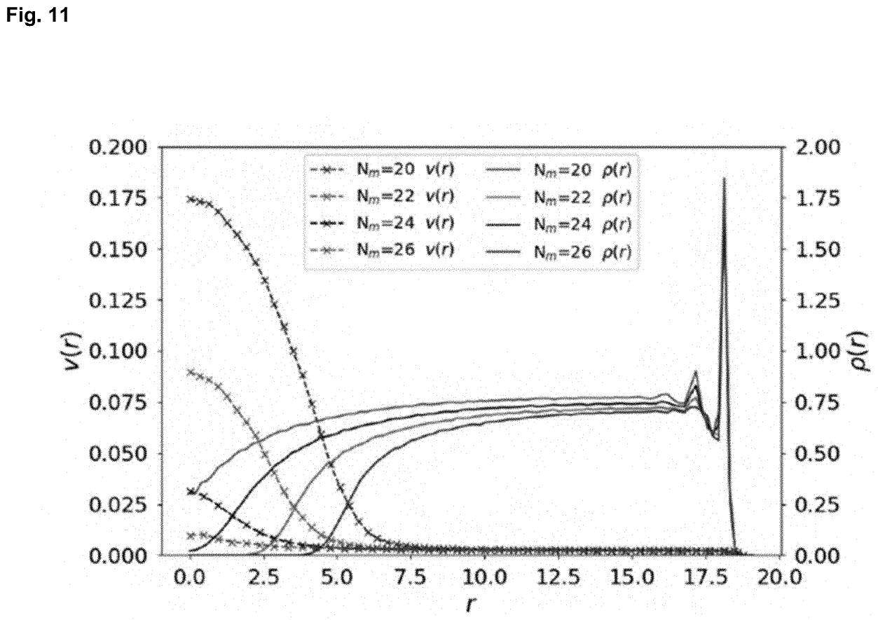

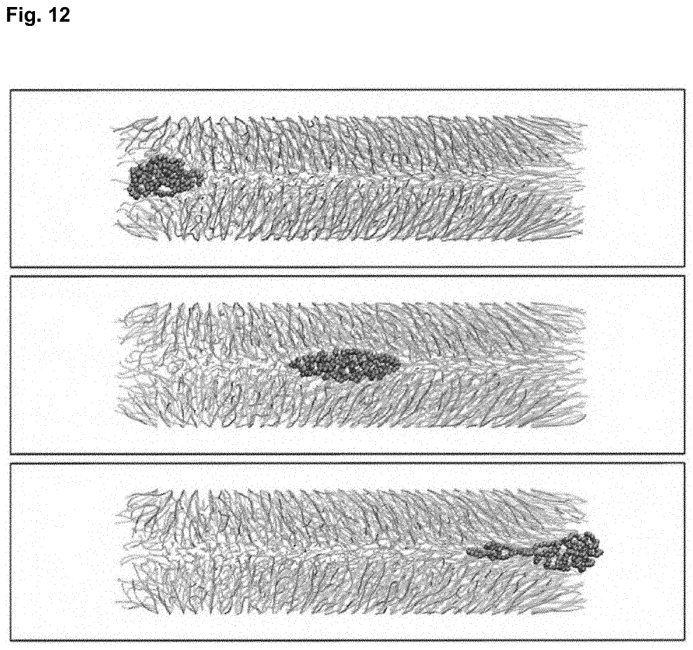

FIG. 12 shows the three main stages, entrance (top panel), traveling (middle panel), and exit (bottom panel) of the typical translocation process in the pore of radius R=9.55σ. The initial deformation of the globular polymer occurs as it enters the pore and is pushed through the monomer density gap against the brush; here the brush polymers are on average pointing against the direction of the flow. As the globular polymer moves through the pore, it is elongated along the cylindrical axis by the radial pressure exerted by the surrounding brush. The conformation of the brush polymers at the end of the pore is on average pointing along the direction of the flow. As the globular polymer exits the pore, it crosses a significant monomer density gradient. While exiting, one part of the globular polymer is in a monomer-free region while the other still feels the radial pressure of the brush. This monomer gradient creates an effective elongational shear at the end of the pore that further destabilizes the conformation of the globular polymer. Once outside the pore, the polymer re-folds into a globular state in a monomer-free environment. FIG. 13 shows the radius of gyration R_{g} as a function of time as the globular polymer translocates through the pore, for multiple translocation events. Here the effect of the pore exit is clearly marked by a sharp peak in R_{g} at the end of each translocation event. Also notice how when increasing the length of the brush, the translocation time increases (top/middle panel), until, when Nₘ=12 the velocity profile inside the pore becomes too weak to drive the globule through it. The figures in the bottom panels show the position of the protein along the axial direction of the pore as a function of time for the different values of Nₘ. Here the x coordinate is propagated and includes the crossing of the periodic boundaries so that the globular protein goes through a series of translocation events over time within the same simulation.

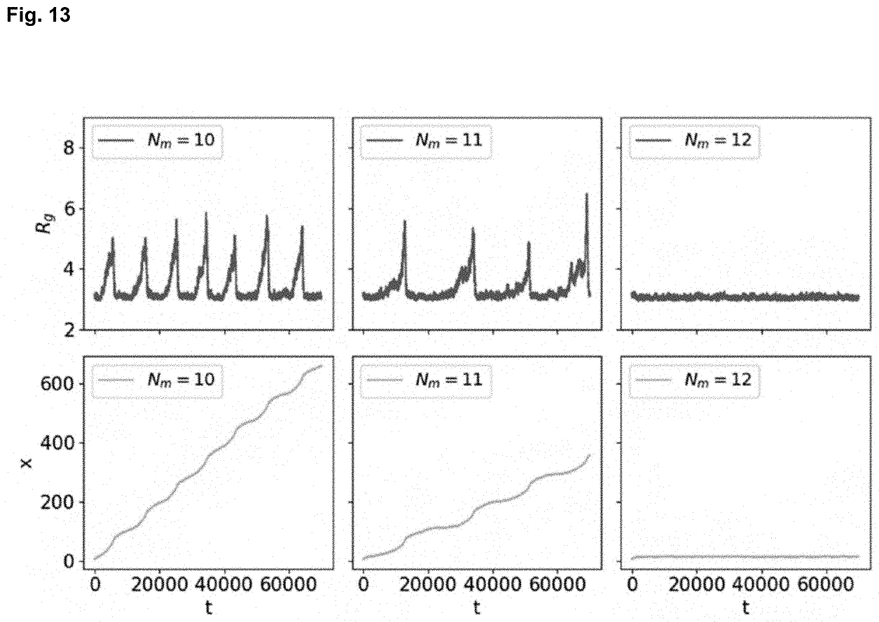

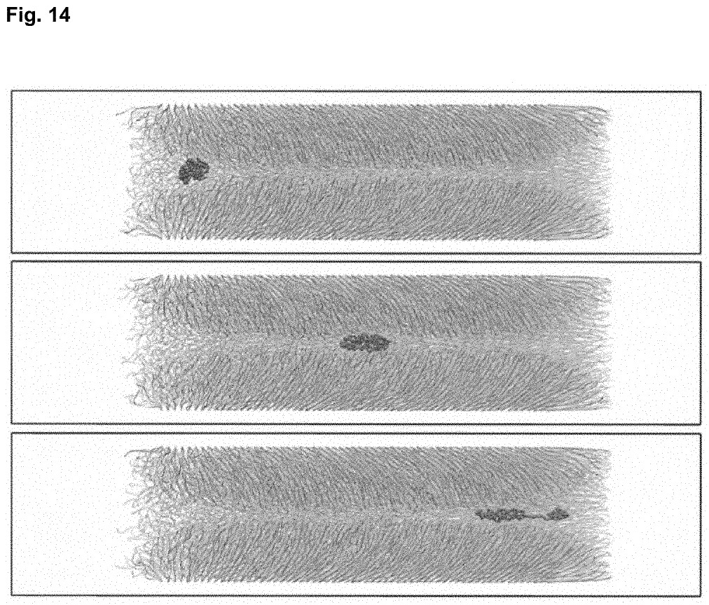

FIG. 14 shows snapshots of the translocation process for the larger Case 3 pore with Nₘ=24. We again see two stages of deformation, at the entrance and at the exit of the pore, and the exiting process is even more dramatic than in the smaller Case 1 pore. The flow velocity for Nₘ=24 in the middle of the pore is more than twice as that for Nₘ=11 in the Case 1 pore with the smaller diameter, suggesting that scaling up the channel has the advantage of being able to translocate larger globules more quickly and generate larger shear forces. Crucially, the larger pore allows for a wider range of brush heights, and corresponding monomer density gaps, to successfully refold the globules. This suggests that larger pores should make more robust re-folders. This is most evident in FIG. 15 (top/right panel) that shows larger R_{g} excursions than those observed for the smaller radius. As expected, the trajectory for Nₘ=20 (top/Left) shows more frequent deformation peaks than those at higher monomer concentration as the velocity inside the pore decreases with Nₘ. While, on the one hand, having a lower monomer density allows for more refolding event, on the other hand the extent of the deformation becomes larger, although less frequent, when increasing the monomer density. Ideally, one would like to set up a system that optimizes these two tendencies.

### Conclusion and Outlook

In this study we considered whether a globular protein, modeled as a globular homopolymer, could be forced to undergo conformational changes when translocating through a cylindrical nanopore internally coated with a polymer brush. We studied the brush profile in the presence of a fluid flow for different values of monomer concentrations, grafting density, and for two pore sizes. Crucially, we find that the influence of the flow on the brush conformation strongly depends on the grafting density of the brush, and when ρ_{G} is sufficiently large, the density profile of the brush is not affected by the presence of the flow, yet the fluid velocity within the pore is very much dependent on the overall monomer concentration (brush height), and drops to small values once the pore becomes completely filled with monomers.

We observe that under the appropriate conditions, high grafting density and sufficiently long chains to leave a small monomer gap along the pore axis, the interaction of the globule with the brush can indeed cause significant deformations of the globule. The globule entrance into and exit from the pore events are of particular interest as they lead to the largest distortions of the globules conformation.

While in this study our protein model, described as a globular polymer, is very rudimentary, tests with a more realistic protein model, retaining some of the specificity of the monomer-monomer interactions that is proper to proteins, are underway and look promising. We believe that the ease with which our protein models deform within the pore is due, in part, also to the large number of intermediate states that can be accessed by the globular homopolymer without a significant loss of interaction energy. For a protein with more specific interactions, the number of low energy misfolded configurations should be much smaller, and the translocation should lead to more significant structural changes throughout the process. Given the large effect of the edges of the pore play in this process, it would be interesting to consider the same process with a polydisperse polymer brush, or with a predefined pattern of brushes of different height. These could be obtained, for instance by mixing two immiscible polymers of different height to promote their phase separation. It is important to stress that our system differs from the GroEL/GroES Chaperonin not only in the origin of the forces driving the possible translocation mechanism (fluid flow vs entropic confinement) but also in the nature of the brush. In fact, it is known that the biopolymers in the equatorial region of the GroEL/GroES complex have hydrophobic ends, which makes it much more likely for misfolded proteins to translocate compared to a correctly folded one. Both simulations and experiments of our system with weakly hydrophobic ends should be feasible and could provide a critical improvement to the purification process.

### Supplementary Material—Conformation of the Brush Under Flow

To quantify the overall behavior of the brush in the presence of the fluid flow, we measured the local brush tilt angle Φ with respect to the cylinder axis along the direction of the flow \vec{ν}=(1, 0, 0). The direction of each polymer in the brush is defined by the vector connecting its grafting point and the last monomer. What follows is the quantitative analysis for the three pores considered in this study.

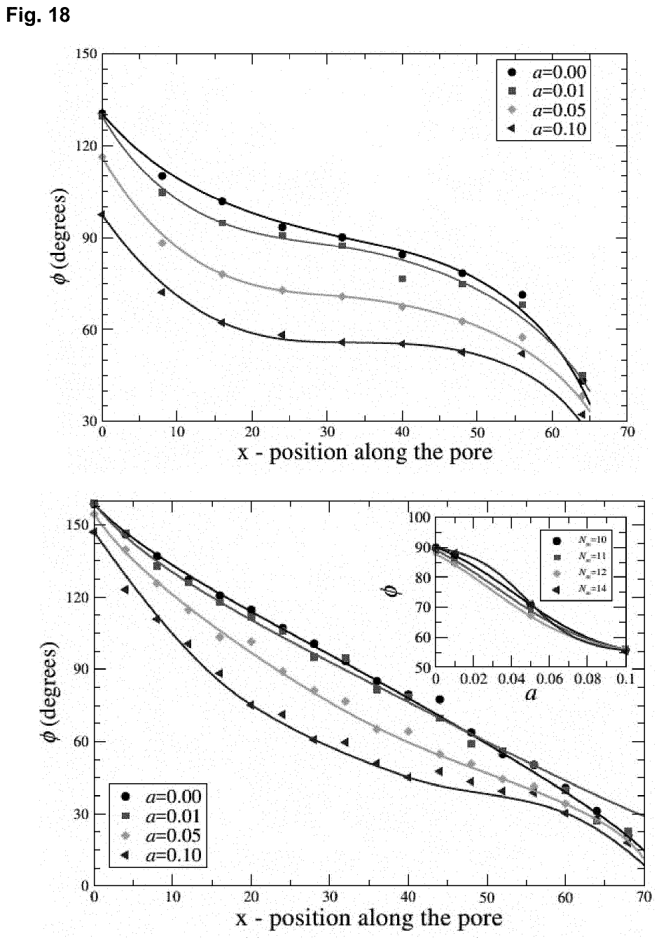

In Case 1, FIG. 18 shows the brush alignment along the length of the pore for flows a=0, 0.01, 0.05, 0.1 and for chain lengths Nₘ=10, 14. Since we expect the middle of the brush (x≅Lₚ/2) to be less sensitive to the behavior of the pore edges, we also show in each inset of FIG. 18 how the tilt angle changes with the fluid acceleration in the middle of the brush for different values of Nₘ. Overall, we observe a systematic decrease of Φ from the equilibrium value of 90 degrees with a. Finally, the insets show how in the middle of the pore, (x≅Lₚ/2), Φ seems to be rather insensitive to the chain length Nₘ.

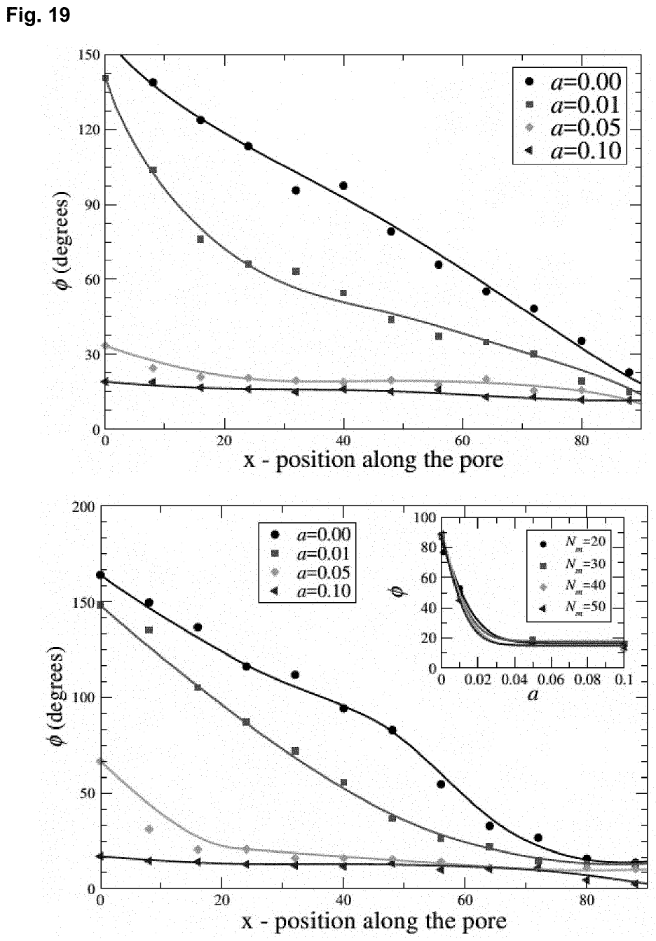

In Case 2, FIG. 19 shows the brush alignment along the length of the pore for flows a=0, 0.01, 0.05, 0.1 and for chain lengths Nₘ=30, 40. Unlike the results for Case 1, at low grafting densities, already for a≥0.05 the brush acquires a clear tilt angle that is roughly constant throughout the length of the pore. As in the previous case, we observe a systematic decrease of Φ from the equilibrium value of 90 degrees with a until it saturates to a value of about 20 degrees. Finally, the inset shows how in the middle of the pore, (x≅Lₚ/2), Φ seems to be rather insensitive to the chain length Nₘ.

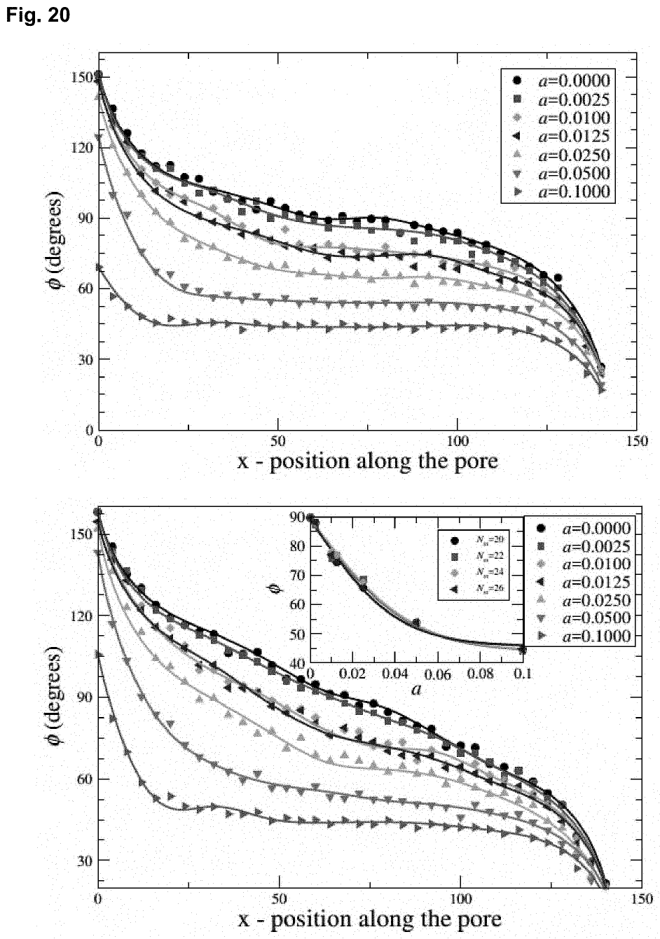

In Case 3, FIG. 20 shows the brush alignment along the length of the pore for flows ranging from a=0 to a=0.1. and for chain lengths Nₘ=20, 24. The front of the pore is characterized by a layer of polymers that resist pointing in the direction of the flow even at the highest driving forces (this effect becomes more evident as one increases Nₘ) while the back side of the brush can easily adapt to the direction of the flow. Overall, even in this case, we observe a systematic decrease of Φ from the equilibrium value of 90 degrees with a. Finally, the insets show how the tilt angle of the brush along the direction of the flow in the middle of the pore changes as a function of a, and again, we observe that Φ is basically independent of the number of monomers in the polymer brush, Nₘ.

### Example 3

### Extrusion Experiments of Bovine Serum Albumin (BSA)

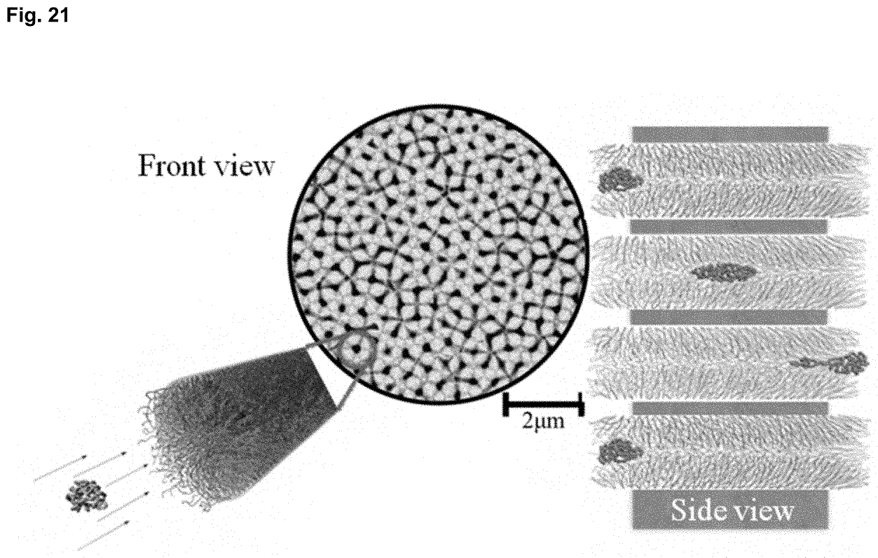

We have performed extrusion experiments of Bovine Serum Albumin (BSA) through the filter prototype (see FIG. 21). The presence of aggregates was assessed using Dynamic Light Scattering (DLS) measurements. The height of the peaks represents the percentage of particles of a given size in the system.

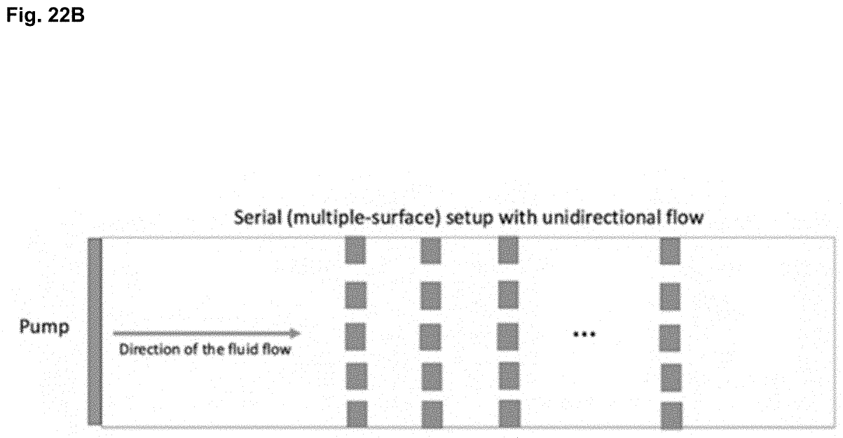

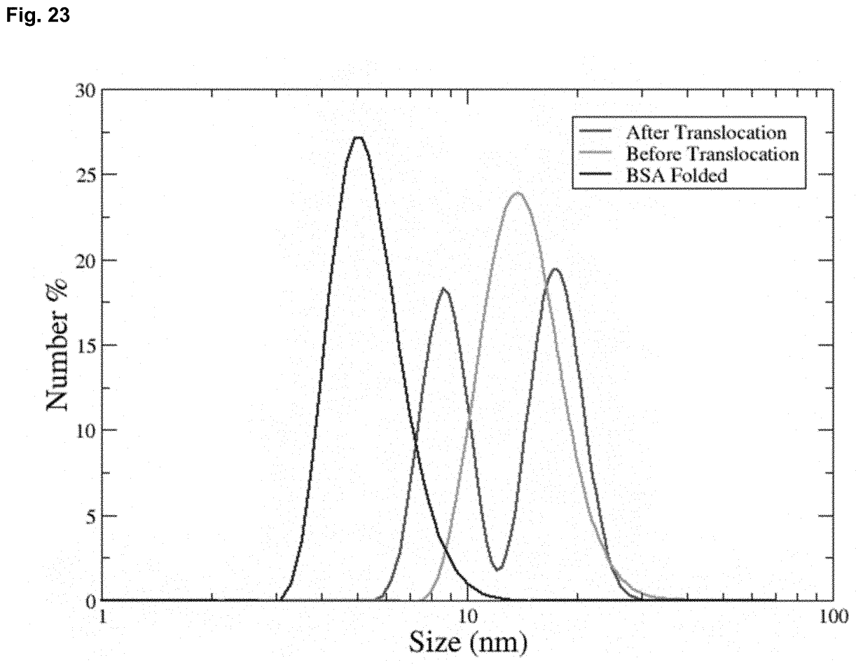

In reference to FIG. 23 and FIG. 24, the BSA has been prepared in three different conditions. The Folded data (in Blue) refers to a correctly refolded BSA assay at the concentration of 4 mg/ml. The Control system (in Green) is a sample at concentration of 10 mg/ml denatured with temperature, and presenting typical aggregates of size ˜14 nm. The Extruded sample (in Red) refers to the typical sizes observed after a single extrusion step (translocation through the pores) of the Control system. The data show, for two different initial distributions (experiments) of the denatured proteins, that already after a single extrusion the aggregates are partially broken bringing the size population closer to the folded one. Crucially in the second experiment, as shown in FIG. 24, the data show that the pore has successfully broken up most of the aggregates to yield a distribution peaked at the same location of the reference folded data (in blue). We believe that the short peak at 2.2 nm in the red curve of FIG. 24 is due to impurities, such as pNIPAM polymers that could have detached from the surface as a result of the fluid flow.

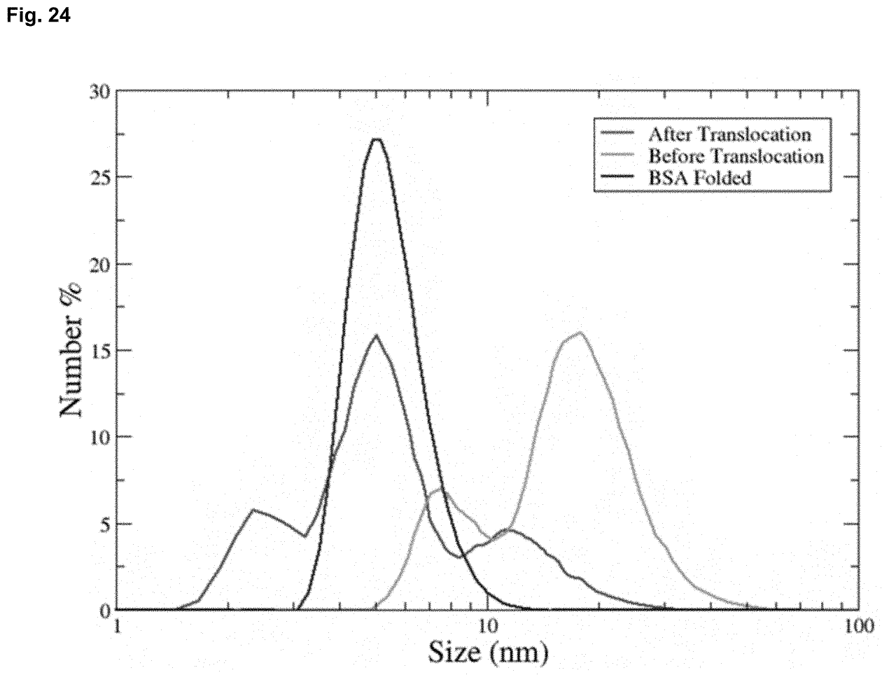

FIG. 25 shows a clearer picture of the size histograms of initially aggregated (A) (red) and native folded (N) (orange) BSA. The blue histogram shows the size distribution after one pass through a chip with ˜150 nm pores functionalized with dense polymer brushes at a concentration of 10 mg/mL. After just a single pass the percentage of refolded proteins has increased by 5-fold (E1). Such performance matches the best efficiency of natural chaperonins that are capable of achieving such improvement with a single pass only in a few cases (de Marco et al. 2007).

### Example 4

### Experiments with Other Proteins

A solution of Green Fluorescent Protein (GFP) is denatured on purpose using the same protocol we used for the BSA. The temperature denaturation causes the GFP to lose their native state and to aggregate. The denatured solution is then pushed through our re-folding device. Upon passage through the polymer coated pores, the aggregates are dissolved, and the proteins refold showing once more their characteristic fluorescence.

The human leukocyte antigen (HLA) is a protein that cannot fold in vitro without the help of molecular chaperones. Unfolded cell extracted HLA is directly translocated through our refolding device. The translocated solution is then tested for refolding using structural antibodies demonstrating the recovery of the folded structure.

Solutions of an antibody for immunotherapy are usually kept at high concentration. These tend to aggregate, and the aggregates reduce the efficacy and the shelf life of the drug. After translocation through the refolding device with one or more passes the antibodies aggregates break apart and the correct population of folded antibodies in solution is restored.

We anticipate carrying out experiments on Lysoszyme (a protein that it is easy to crystallize if properly folded and will allows to make a full structural biology study to measure the refolding efficiency), and other recombinant proteins that are top of the line in the pharma industry, including but not limited to: Rituximab, Eculizumab, rHGH, rFVIIa, rHepatitis B Surface Antigen, and rFVIII (Puetz and Wurm, 2019) to further validate and extend the findings disclosed herein.

### Example 5

### Smart Nanopores to Detect and Refold Misfolded Proteins and Aggregates

### Introduction

Protein misfolding and the subsequent formation of aggregates is a major problem both in the human body and in the laboratory. In vivo, misfolding is responsible for a range of neurodegenerative diseases, including Parkinson's, Alzheimer's, and Creutzfeld-Jakob diseases. In vitro, misfolding and aggregation can dramatically lower the yield in recombinant protein synthesis. In the body, specialized molecular structures called chaperones are responsible for sequestering and refolding misfolded proteins. In the laboratory, several techniques have been developed to destroy misfolded aggregates. In general, these techniques require addition of a cocktail of buffers and chaotropes. In one method, high osmotic pressures are applied, changing the protein thermodynamics, and the proteins de-aggregate. In another method, a vortex fluid device is used to apply large shear forces to the protein and tear apart the aggregates. Both methods require careful optimization not only of the solvent, but also the protein concentration and processing time. In addition, existing methods require a prior purification step to first separate the native and misfolded proteins.

Here, we present a smart nanopore that both identifies and refolds misfolded proteins. Pressure-driven flow of protein solutions through a nanopore with hydrophobic surfaces allows for selective refolding of the misfolded proteins, and this device greatly simplifies purification by combining purification and refolding into a single step. This smart nanopore leverages two physical principles: shear forces due to the solvent flow profile and protein adsorption on hydrophobic surfaces.

As mentioned, shear forces can be used to destroy protein aggregates (Yuan et al. 2015). Previous studies have also shown that shear also distorts and unfolds individual proteins (Alexander-Katz et al. 2006; Alexander-Katz and Netz, 2008; Schneider et al. 2007). Alexander-Katz and coworkers studied the shear-induced unfolding of von-Willebrand (vWF) factor proteins, and by modeling blood flow within blood vessels as a constant shear and the protein as a polymer globule, showed that unfolding occurs above a critical shear rate. Jendrejack and coworkers studied the dynamics of flexible DNA flowing in microchannels and incorporated wall-DNA hydrodynamic interactions (Jendrejack et al. 2002; Jendrejack et al. 2004; Jendrejack et al. 2003), and showed that due to the parabolic solvent velocity profile, the DNA stretches more near the walls, where the shear rate is high, and stretches little at the channel center, where there is no shear. They also showed at higher solvent velocities, the DNA tends to remain near the channel center and away from the walls.

Protein adsorption on surfaces has been extensively studied. Whitesides showed that the adsorption rate can be controlled by tuning the hydrophobicity of self-assembled organic monolayers (Prime and Whitesides, 1991). Others have shown that surface adsorption distorts protein conformations (Roach et al. 2005). Indeed, proteins can even adsorb onto polymer brushes (Roach et al. 2005). In every case, attractive interactions between hydrophobic residues and surfaces cause proteins to adsorb and swell.

Finally, protein folding to the native state is largely driven by burying hydrophobic residues in the interior. When proteins fail to do so, this results in a misfolded structure with a larger fraction of hydrophobic residues on the surface, and it is these “greasy” surfaces of misfolded proteins that further drive protein aggregation. Therefore, misfolded proteins and aggregates will experience a larger attraction to a hydrophobic surface. Furthermore, it has been shown that hydrophobic environments can enhance protein folding, operating on the same principle as the hydrophobic interior of a chaperone (Jewett et al. 2004; Jewett and Shea, 2006).

With this mind, the operation of the smart nanopore is as follows: native proteins will flow on unperturbed in the nanopore center, while misfolded proteins will be attracted to and captured by the hydrophobic walls, where the solvent shear velocity is the highest. Then, a combination of adsorption and shear will unfold the misfolded proteins. We demonstrate the efficacy of the nanopore with theory and simulations.

### Methods

Our model describes the protein as a fully flexible polymer of N beads of radius a and mass M connected by harmonic springs. The springs have the potential Vₛ(r)=Kₛ(r−r₀)², where Kₛ=200k_{B}T and r₀=1.0σ. Beads interact via the Lennard Jones (LJ) potential

$$V ( r ) = 4 ɛ [ {( \frac{σ}{r} )}^{12} - {( \frac{σ}{r} )}^{6} ] ,$$

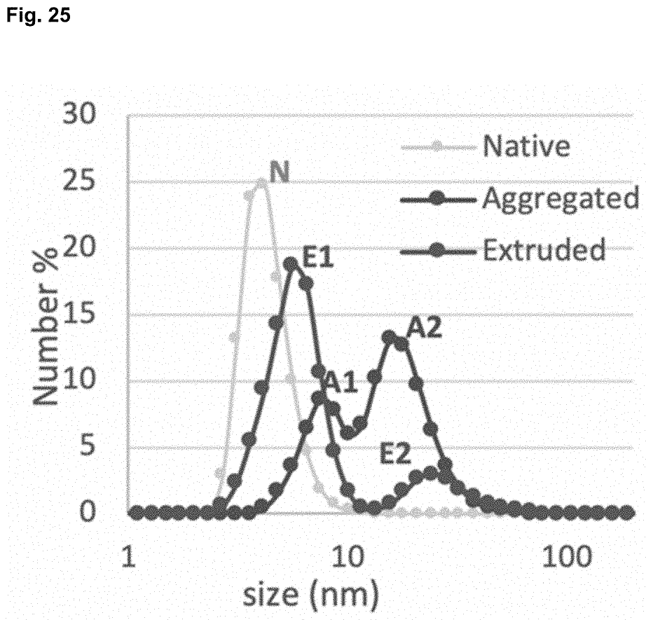

which is cut at 2.5σ. The cylindrical wall interacts with the polymer with the same LJ potential, with σ_{ω}=0.5σ. For the repulsive wall, the potential is cut at 2^{1/6}σ_{ω}. For the attractive wall, the potential is cut at 2.5σ_{ω}. Following Alexander-Katz and coworkers (Alexander-Katz et al. 2006) (FIG. 26), we set the bead attraction ε=2.08k_{B}T, which yields a strongly collapsed polymer globule at equilibrium.

We use a Multiparticle Collision Dynamics (MPCD) solvent that correctly accounts for the solvent flow profile and polymer bead-bead and bead-wall hydrodynamic interactions. MPCD is a particle-based algorithm for solvent hydrodynamics that can easily be coupled to solute molecular dynamics. MPCD consists of alternating streaming and collision steps.

The streaming step consists of updating the positions:

xᵢ^{t+1}=xᵢ^{t}+vᵢΔt (5)

In the collision step, particles are sorted with a grid of size a, and the velocities are updated according to:

vᵢ^{t+1}=u_{ξ}^{t}+R(vᵢ^{t}−u_{ξ}) (6)

Here, u_{ξ} is the center-of-mass velocity of all particles within bin ξ, and R is a stochastic rotation matrix.

We choose our solvent parameters to match those of previous MPC studies of polymers in microcapillary flows (Chelakkot et al. 2010), and they are as follows: the solvent timestep is Δt=0.1τ, the stochastic rotation angle π/2, the average solvent particles per bin ρ=10, fluid mass m=M/ρ, the fluid mass density [symbol] =μm/a³, the grid size a=σ, and the MD timestep Δt_{MD}=5×10⁻³τ. The nanopore radius is R=10σ, and we study various pore lengths from L=50σ to 100σ. Pressure-driven flow is achieved by applying a constant force g in the axial z direction to all fluid particles, and the measured velocity profiles agree with the analytical solution to the Stokes equation.

All quantities are multiples of the fundamental units k_{B}T=1, τ₀=\sqrt{ma²/k_{B}^{T}}, and g₀=k_{B}T/a. All simulations were run for a minimum of 4×10⁷ r time steps. All simulations are performed with the LAMMPS molecular dynamics software package.

### Results and Discussion

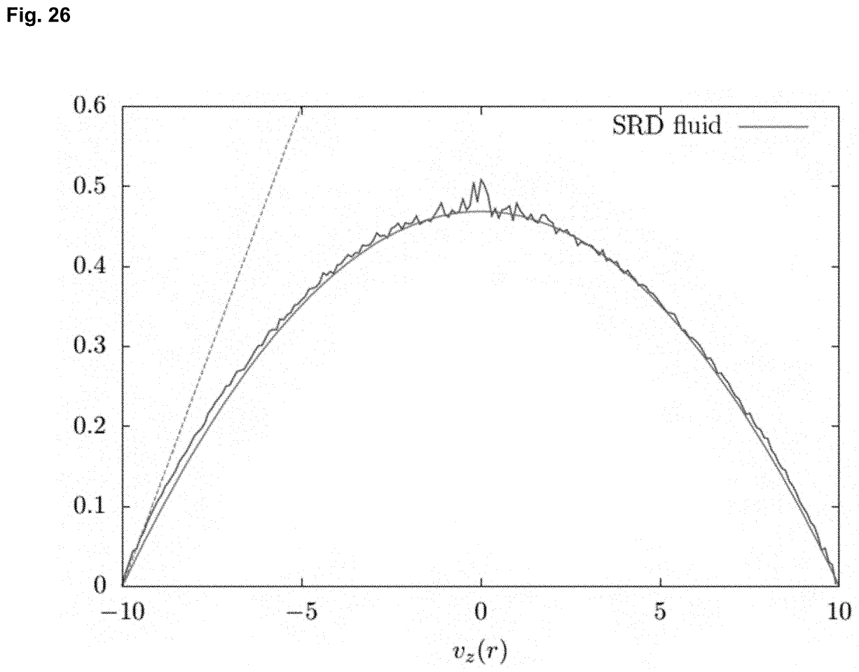

A structural diagram for a range of forces g and wall hydrophobicities ε_{ω} is presented in FIG. 27, showing the average end-to-end distance <R_{z}> along the cylindrical axis for each set of parameters. At low forces and ε_{ω}=1, the polymer remains in a globular state. For g=0.012 and ε_{ω}=1, stretch-and-unfold events begin to occur. Analysis of trajectories shows that unfolding occurs far from the wall, where there is zero bead-wall interaction, so unfolding is purely due to solvent flow induced shear. We note that at g=0.012, if one approximates the solvent flow profile as a constant shear, the shear rate is roughly half that of Alexander-Katz's critical shear rate. Therefore, the curvature of the solvent flow profile enhances unfolding.

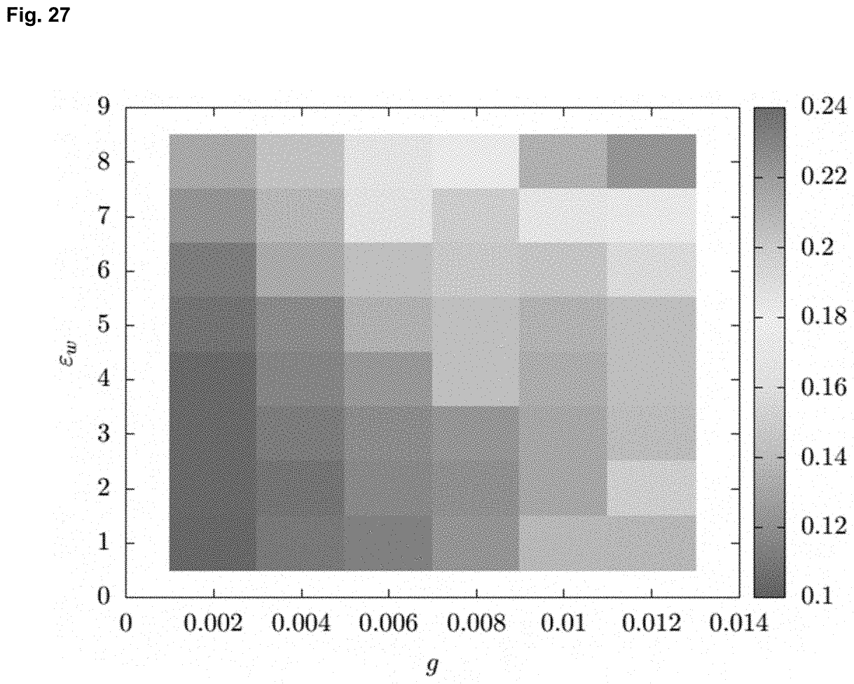

As ε_{ω} increases, globules become attracted to the wall. FIG. 28 shows the axial extension R_{z}/Nₐ of the polymer along trajectories for different wall hydrophobicities. At ε_{ω}=5.0, the polymer begins to attach to the wall and several unfolding events occur. At ε=7, many unfolding events occur. At these values of c, the polymer can still detach from the wall. However, at e=8, the polymer is completely bound to the wall, and this stabilizes the polymer conformations. The same trend is seen for g=0.006.

As noted by Jendrejack and coworkers, at increasing flow velocities, polymers become localized to the channel centerline, and this can be thought of as an effective hydrodynamic repulsion from the wall. Though the frequency of reaching the wall is diminished at higher velocities, once the polymer is captured, unfolding can occur.

### Conclusions

The results disclosed herein show that forced flow of dilute protein solutions in a nanopore with hydrophobic walls can be used to capture and unfold misfolded proteins, and we have shown parameters under which successful unfolding occurs.

We continue to apply polymer theory to explain how protrusions form under shear flow, which is within the scope of the present disclosure. As noted by Alexander-Katz and coworkers, a polymer globule is constantly rotating under shear forces, and because of this, short protrusions are immediately wound back into the globule. How the attractive wall affects rotations of the globule and its effects on globule unfolding are part of the present disclosure and are continuing to be pursued.

### Example 6

### Soft Nanopores for Refolding Proteins

### Introduction

A protein's function is determined in large part by its sequence of amino acids and its folded configuration. The latter is often unique for a given sequence and the protein function is tightly coupled to the correct realization of the native structure. There are many catastrophic events that can take place when just a few proteins fail to reach their functional configuration (Dobson et al. 1998). In particular, a major obstacle along the correct folding pathway of a protein is represented by potential aggregation with other copies of the proteins. Such aggregates can form precipitates in the in vitro refolding experiments dramatically reducing the yield, or in vivo the formation of large clusters can be lethal to the cells and in the long term cause serious diseases such as Alzheimer or Parkinson's (Dobson et al. 1998; Martin and Hartl, 1997; Hoang et al. 2006; Combe and Frenkel, 2007; Orte et al. 2008; Zhou et al. 2009; Uversky, 2011; Schor et al. 2012; De Simone et al. 2012; Giannozzi et al. 2012; De Santis et al. 2015; Castello et al. 2015; Shimanovich et al. 2015). Hence, misfolding and subsequent aggregation can pose a significant risk to all living organisms. In light of this risk, evolutionary pressure has developed complex protection mechanisms against misfolding. For instance, in prokaryotic cells the GroEL/GroES chaperonin complex acts as an effective protection against misfolding and aggregation (Sigler et al. 1998; Martin, 2002; Burston et al. 1996; Falke et al. 2005; Weber et al. 1998; Ricci et al. 2016; van der Vaart et al. 2004). The GroEL/GroES chaperonin is a double barreled complex with two large cavities, in which misfolded proteins are captured and isolated for a long time 15 s) and at considerable energy cost (7 ATPs per protein or 14 per cycle). The working principle of the GroEL/GroES has not been fully elucidated, but the consensus is that its main function is to segregate misfolded proteins from the cytosol and into a molecular cage, and in this way, completely avoid aggregation. Secondly, the GroEL complex helps the trapped proteins to escape from misfolded configurations. (van der Vaart et al. 2004; Coluzza et al. 2006; Sirur and Best, 2013; Jewett et al. 2004). Recently Coluzza et al. have postulated an additional reaction pathway for the GroEL/GroES complex that involves translocation through the equatorial region that connects the two barrels (Coluzza et al. 2006; Coluzza et al. 2008). In this work, it was demonstrated that translocation through a narrow pore is an efficient method to help proteins leave local minima regardless of their sequence of amino acids. Although such a pathway has not yet been experimentally investigated, it offers an interesting design principle for a device that could be used in vitro to promote the correct folding pathway and bypass the aggregation pitfalls.

Using computer simulations, we find a soft cylindrical pore, internally decorated with a polymer brush, as an optimal design for an artificial chaperon. Our results have been obtained with computer simulations of off-lattice protein models that have been used in the past to elucidate the refolding action of the GroEL/GroES chaperonin (Jewett et al. 2004; Baumketner et al. 2003). As a proof of concept, we show how misfolded proteins and aggregates driven by a flow through the soft nanopore are easily forced to break apart and unfold, thus allowing a second chance to refold in to the correct native state. In contrast to small slits or small diameter hard nanopores, the larger soft pore is not clogged by the proteins. Forced translocation through the grafted polymer brush disrupts the protein structure and subsequently unfolds it. Aggregate-breaking occurs in a similar fashion. As aggregates are forced through the nanopore, single proteins are peeled off by steric interactions with the brush, resulting in separated proteins. This allows the proteins to refold in condition similar to isolation conditions.

The working principle of our artificial chaperone is significantly different from shear based unfolding methods, because the latter is known to promote aggregation under the effect of the flow (Bekard et al. 2011), while the artificial chaperon works exactly in the opposite direction preventing aggregation. Recently, Yuan et al. proposed an experimental protocol to enhance the refolding of several proteins using a combination of shear and urea (Yuan et al. 2015). Their results show successful refolding and dis-aggregation of clusters of several proteins. A shortcoming of their technique is that their experimental parameters need to be tailored to the specific protein treated. Conversely, an advantage of the soft nanopore is that the designed geometrical properties of the cylindrical pore should guarantee the proper refolding activity for a wide range of protein sizes and cluster sizes regardless of the specific sequence of the protein.

### Results

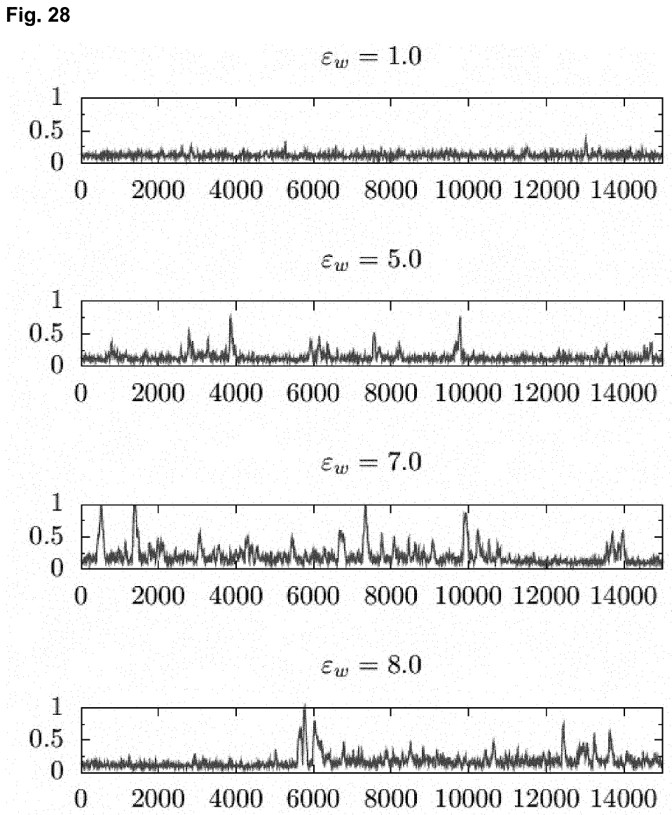

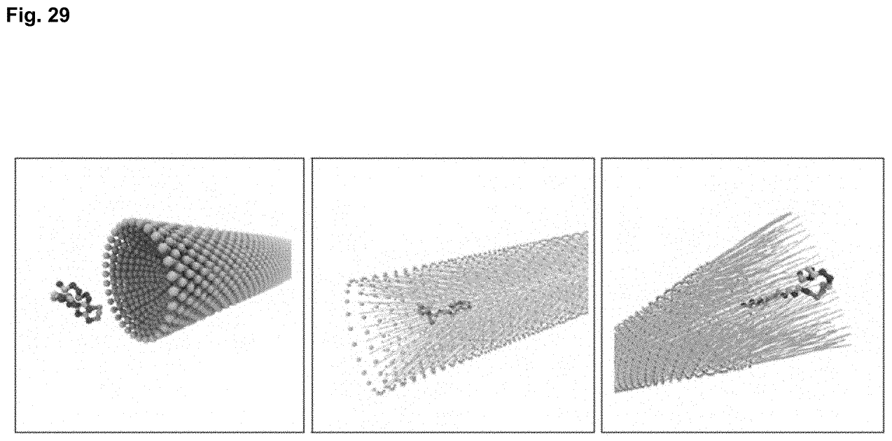

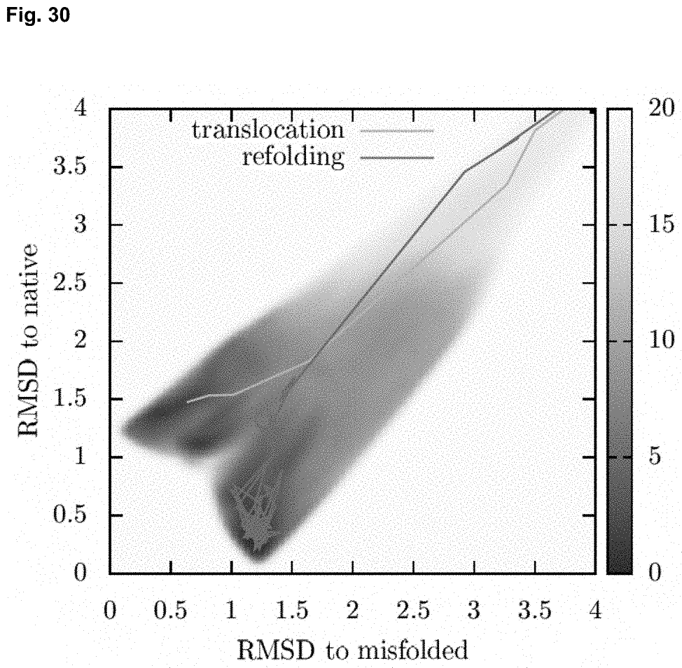

First, we studied the translocation of single misfolded proteins through the soft nanopore. We examined two frustrated proteins described by different off-lattice, coarse-grained models. Both are two-state proteins with a metastable and native state. One is a 27 residue HP model α-β sandwich protein introduced by Jewett et. al, which we call the Shea protein. The other is a 54 residue GO-model protein of the 2GYC-X segment with only short-ranged interactions, which we call the Coluzza protein. The grafted cylindrical polymer brush is described by tethered, fully flexible chains of hard beads. The grafting density, cylinder radius, and cylinder height and the chain length of the grafted polymers describe the polymer brush. Unless specified otherwise, the grafting density is 1.7σ, the cylinder has radius r_{cyl}=8.5σ and height h_{cyl}=60σ. Both protein beads and polymer beads have the same diameter a, and the chain lengths are reported as the number of beads along one polymer. Snapshots of the system are shown in FIG. 29. The free energy landscapes of the two proteins are shown in FIG. 30 and FIG. 31, and barriers of more than 10k_{B}T separate the native and misfolded states, ensuring that spontaneous transitions between the two states do not occur. Protein translocation is achieved by a constant force on every protein and polymer brush bead, modeling pressure-driven flow.

Individual translocation trajectories through nanopores with chain length 13 and driving force 8 show that the protein is forced to unfold when it is pushed through the brush-coated nanopore. The root mean squared deviations (RMSD) of configurations along translocation trajectories in FIG. 30 and FIG. 31 show that the nanopore forces the protein to explore regions of configuration space extremely far from the native and metastable basins of attraction and to adopt structures with free energies greater than 20k_{B}T. Once the unfolded protein exits the brush-coated pore, it is able to refold. Snapshots of a typical translocation trajectory are shown in FIG. 29.

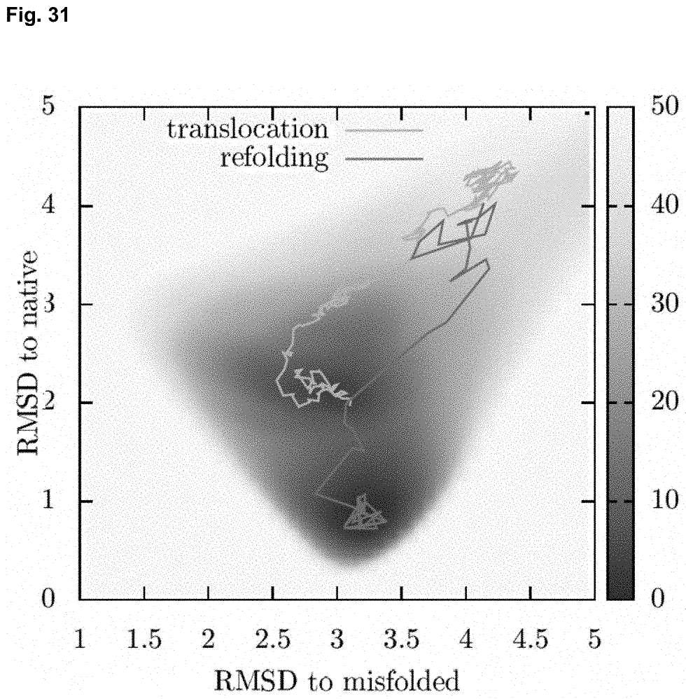

To quantify the refolding, we performed 200 translocation trajectories of metastable Shea and Coluzza proteins through nanopores with brush polymer chain length 13, and driving force 8, and units are given in the Methods section below. The RMSDs of the proteins before translocation, and after translocation and refolding, are shown in FIG. 32. After translocation and refolding, a significant fraction of native state proteins are recovered. Inspection of configurations along translocation shows that in order to enter the nanopore, the proteins must first unfold and adopt a compact, linear geometry, as shown in FIG. 29. After exiting the nanopore, the proteins are then able to refold. We note that post-translocation, folding into the native state is not guaranteed, and it is the underlying protein sequence and energetics that determine the fraction of native state proteins obtained.

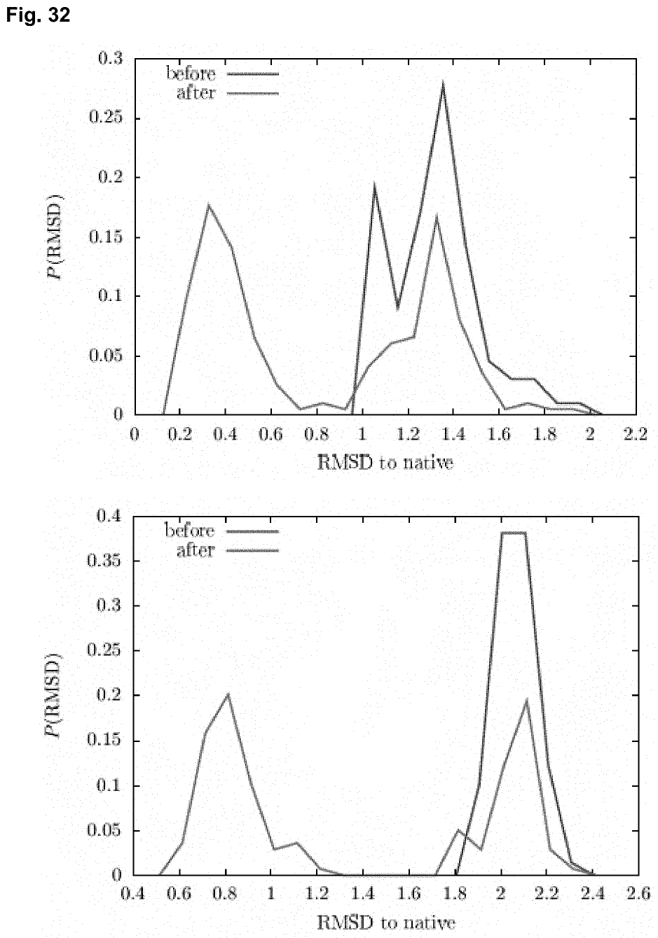

To better understand the unfolding action of the soft nanopore, we explored combinations of different translocation forces and polymer chain lengths for a nanopore of radius 8.5σ. 20 trajectories of the Shea protein were driven through the nanopore for every combination of parameters, and the results are shown in a structural diagram in FIG. 33. The diagram reveals a large region of parameter space for which the soft nanopore can unfold the protein. At small chain lengths, the protein successfully translocates and is unaffected by the nanopore. At large chain lengths, the protein cannot enter the pore, corresponding to a clogged nanopore. However, at chain lengths 12-14, the protein is able to enter the pore, and the combination of the driving force and steric interactions with the polymer beads unfolds the protein.

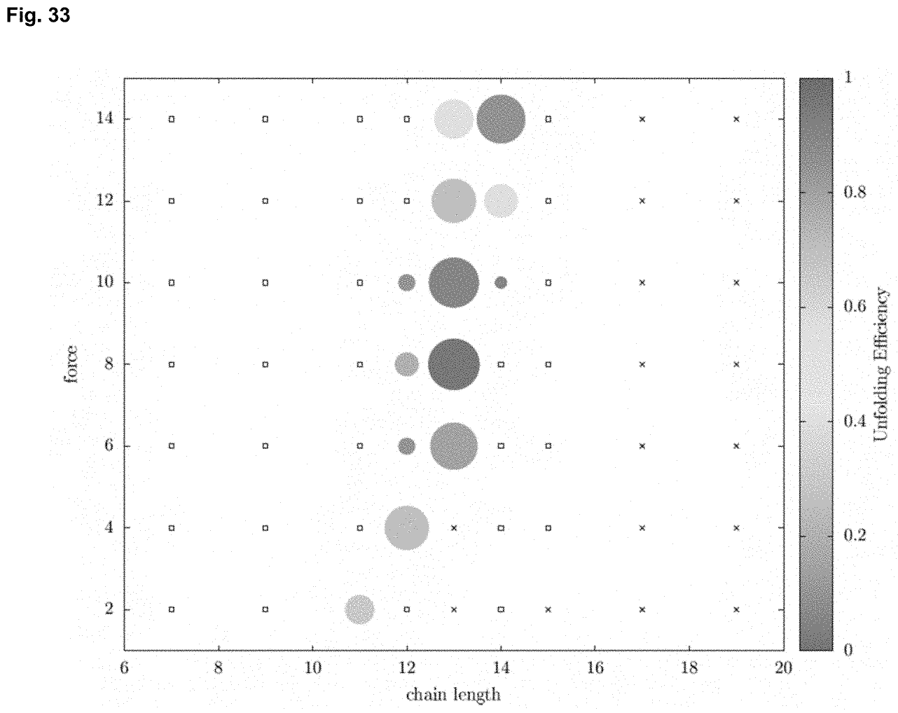

Proteins that unfold are always driven through the nanopore center, and this suggests that the monomer density profile of the polymer brush plays an important role in unfolding proteins. Plots of radial monomer density profiles of the grafted polymers are shown in FIG. 34 (top) for pores with different chain lengths and driving forces. The density profiles correlate the unfolding success rate to the polymer chain length. At short chain lengths, there is a large density gap at the pore center, and the proteins pass through the pore unperturbed. At large chain lengths, the nanopore is completely filled with polymer beads, completely blocking proteins from entering the pore. At intermediate chain lengths, there is a density gap at the pore center, and these are the parameters that give the highest unfolding efficiency (see FIG. 33). This density gap plays the role of an effective pore size, and when the density gap is 4σ, or several residues wide, translocation then results in protein unfolding. Sharper curvature of the density gap corresponds to higher unfolding efficiency. Therefore, the chain length of the grafted polymers determines the monomer density profile, which creates an effective pore size, and this pore size corresponds to the unfolding efficiency.

FIG. 33 also shows that the unfolding efficiency also depends on the driving force, and plots of the monomer density profiles in FIG. 34 (bottom panel) help to elucidate this. At small driving forces, the chains stretch towards the nanopore center, as with a standard polymer brush. At large driving forces, polymer chains tilt towards the nanopore walls and are stretched axially along the flow direction. This tilting of the polymers increases the density gap at the center of the pore. FIG. 34 (bottom panel) shows how the driving force affects the width and curvature of the density gap. Again, smaller widths and sharper curvatures of the density gap correspond to higher unfolding efficiency. In addition to the polymer chain length, the driving force also affects the monomer density gap, and is a second control parameter that determines the unfolding efficiency.

As shown in FIG. 32, at the best combination of force and nanopore chain length, the Coluzza protein also achieves similar unfolding success rates. This is a remarkable result, as the Coluzza protein is comprised of a completely different model and has very different energetics. Furthermore, the Coluzza protein is larger than the Shea protein, having twice as many residues as the Shea protein. This shows that the unfolding action of the soft nanopore is independent of both the sequence and size of the protein, and is principally determined by the monomer density profile. If the density gap in the monomer density profile is smaller than the size of a protein, translocation through the nanopore will unfold the protein.

Unfolding proteins is the principal function of the soft nanopore, and as shown in FIG. 32, depending on the underlying thermodynamics of a protein, a significant fraction of metastable proteins may be recovered after translocation. However, repeated translocation through the nanopore will increase the yield of native proteins.

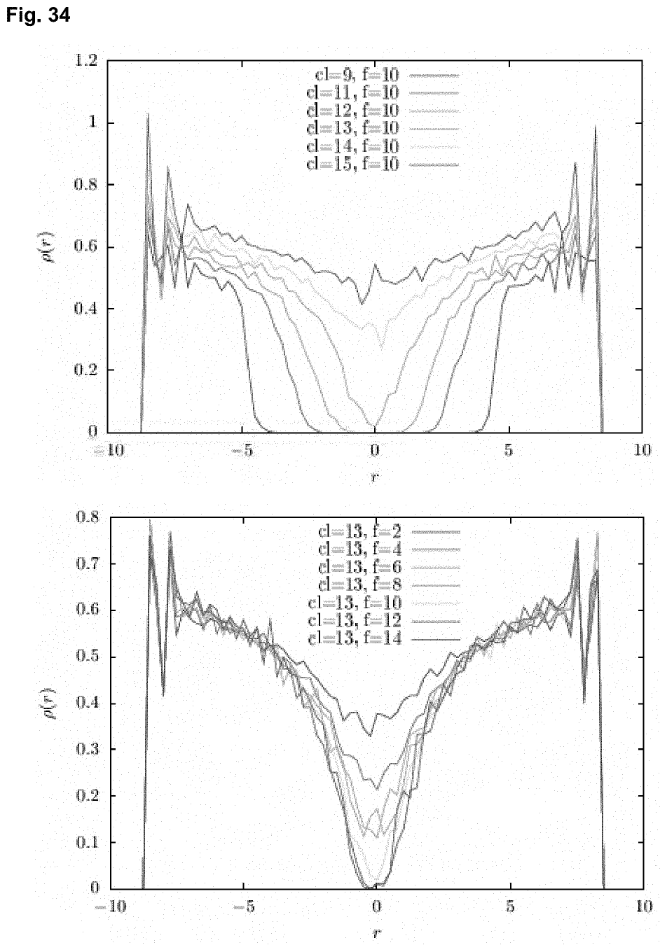

We now turn to aggregates, or clusters of proteins. Protein aggregates, as collections of metastable proteins bound to one another, can be viewed as a metastable state in and of themselves. We demonstrate that the forced translocation through the soft nanopore can break apart protein aggregates (FIG. 35). Cofolding the Shea protein at high densities results in a dimer. Out of 100 translocation events for a soft nanopore with chain length 13 and driving force 8, 91 dimers are broken and unfolded, resulting in pairs of proteins separated by a distance of at least 10σ. This separation distance of the proteins after translocation is crucial in allowing the proteins to refold independently and to prevent re-aggregation.

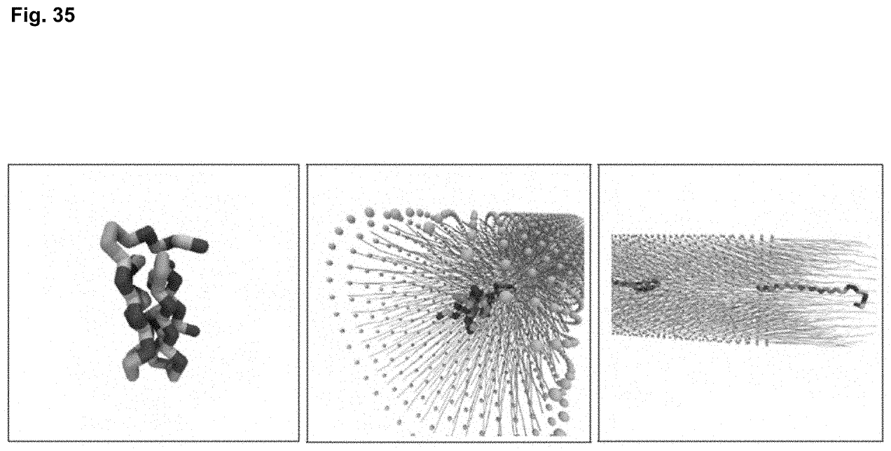

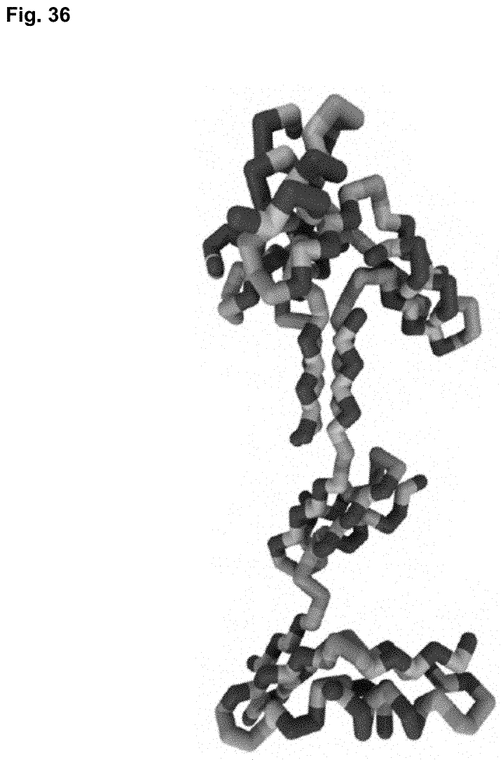

We also created a 9-mer aggregate by co-folding the Shea protein at extremely high concentrations, which can be viewed as an example of a kinetically trapped, metastable state (FIG. 36). We emphasize this is a worst-case scenario for aggregation, as cofolded aggregates are much more strongly bound than aggregates of already-folded proteins. We used a larger nanopore with r_{cyl}=12σ, h_{oyl}=93σ, grafting distance 1.7σ, chain length 19, and driving force 8. Translocation under these conditions breaks the 9-mer and results in single separated proteins and several entangled proteins (FIG. 37). Though entangled proteins can refold into aggregates, repeated forced translocations through the soft nanopore will eventually break the smaller aggregates into individual proteins.

### Methods

All simulations were produced using the LAMMPS software package. We model the cylindrical brush as fully flexible chains of WCA particles with diameter a connected with harmonic springs with the potential V(r)=K (r−r₀)², with K=200k_{B}T and r₀=σ. Grafted particles are arranged in a hexagonal lattice with lattice spacing d₀=1.7σ, at a distance r_{oyl} from the cylinder center. An additional cut-and-shifted 12-6 Lennard Jones repulsive wall is placed at r_{cyl}.

The Shea protein is a one-bead model with hydrophilic, hydrophobic, and neutral residues that interact via a generalized Lennard-Jones potential

$$V ( r ) = 4 ɛ ( {( \frac{σ}{r} )}^{1} 2 - {Λ ( \frac{σ}{r} )}^{6} ] .$$

Beads are connected with harmonic springs of the form V_{pb}(r)=200k_{B}T(r−σ)² and angles of the form V_{ba}(r)=40k_{B}T(θ−θ₀)², where θ₀=1.8326 in radians.

The 27 bead sequence of the Shea protein is: (LB)₃N₂(BL)₃N₃(B₂L₂)₂BL.

The interaction matrix for ϵ is:

| | B | L | N |
|---|---|---|---|
| B | ϵₕ | $\frac{7}{12} {ϵ}_{h}$ | $\frac{2}{3} {ϵ}_{h}$ |
| L | $\frac{7}{12} {ϵ}_{h}$ | $\frac{1}{6} {ϵ}_{h}$ | $\frac{1}{4} {ϵ}_{h}$ |
| N | $\frac{2}{3} {ϵ}_{h}$ | $\frac{1}{4} {ϵ}_{h}$ | $\frac{1}{3} {ϵ}_{h}$ |

The interaction matrix for A is:

| | B | L | N |
|---|---|---|---|
| B | 1 | 0 | 0 |
| L | 0 | −1 | 0 |
| N | 0 | 0 | 0 |

The 24-angle dihedral sequence gives rise to α-helices, 3-sheet regions, and turns. Starting from the N-terminus, the sequence is: β₄T₃β₅T₂α₁₀. The dihedral potential is:

$${V}_{ta} ( φ ) = - A {cos}^{6} ( \frac{φ - {φ}_{α}}{2} ) - B {cos}^{6} ( \frac{φ - {φ}_{β}}{2} )$$

where Φ_{α}=1.0 rad and Φ_{β}=π. For α-helices, A=6ϵₕ, B=5.6ϵₕ. For β-sheets, A=5.6ϵₕ, B=6.0ϵₕ. For turns, A=B=0.

Our Coluzza protein is a GO model representation of the X domain of the 2GYC protein. Residues are represented by single beads and have repulsive WCA interactions for excluded volume, and successive beads are connected with the same harmonic bonds as the brush polymers. An Gō-type interaction matrix is created by reading the PDB structure, then using the coordinates of the Cc, atoms as the minimum for Gaussian wells with well depth ϵ_{G}=2k_{B}T and variance 2. The Coluzza protein only has short-ranged interactions, such that pairs of residues further than 4σ do not interact.

Langevin dynamics were used for both the translocation simulations and for the free energy calculations, with a damping coefficient of 0.1. Timesteps are given in units of τ₀=\sqrt{mσ²/ϵ}. The translocation driving force is given in units of k_{B}T/σ.

Free energy landscapes were calculated using the well-tempered metadynamics method. 4 replicas were used and hills with weight 0.05 and width 0.1 were deposited every 2000 timesteps, with bias temperature 10, and the landscapes were checked for convergence. To produce the reference native and metastable configurations, folding trajectories of a minimum 10⁷ timesteps were initiated, then we minimized the energies of the resulting configurations.

### Conclusions

In this Example, we have shown how a forced translocation of proteins through a soft nanopore can unfold misfolded proteins and destroy aggregates. The driving force and the brush polymer chain length determine the density gap, and the depth and curvature of this density gap are responsible for the unfolding efficiency of the pore. We have shown successful unfolding for two different models and several different aggregates, showing that the nanopore works for a range of protein sequence and sizes. Work is currently underway to systematically characterize the breaking efficiency of translocation for different sized aggregates.

### DOCUMENTS CITED

The following references, to the extent that they provide exemplary procedural or other details supplementary to those set forth herein, are specifically incorporated herein by reference.
1. A. Alexander-Katz and R. Netz, Macromolecules 41, 3363 (2008).
2. A. Alexander-Katz, M. Schneider, S. Schneider, A. Wixforth, and R. Netz, Physical review letters 97, 138101 (2006).
3. A. Baumketner, A. Jewett, and J. Shea, Journal of molecular biology 332, 701 (2003).
4. A. De Simone, C. Kitchen, A. H. Kwan, M. Sunde, C. M. Dobson, and D. Frenkel,

Proceedings of the National Academy of Sciences 109, 6951 (2012).
5. A. Jewett and J.-E. Shea, Journal of molecular biology 363, 945 (2006).
6. A. Jewett, A. Baumketner, and J.-E. Shea, Proceedings of the National Academy of Sciences of the United States of America 101, 13192 (2004).
7. A. Lamura, G. Gompper, T. Ihle, D. M. Kroll, EPL (Europhys. Lett.) 56 (2001) 319. ISSN 0295-5075.
8. A. Orte, N. R. Birkett, R. W. Clarke, G. L. Devlin, C. M. Dobson, and D. Klenerman, Proceedings of the National Academy of Sciences 105, 14424 (2008).
9. A. Sirur and R. B. Best, Biophysical journal 104, 1098 (2013).
10. A. van der Vaart, J. Ma, and M. Karplus, Biophysical journal 87, 562 (2004).
11. A. L. Yarin, J. Non-Newtonian Fluid Mech. 37 (1990) 113.
12. A. R. Kinjo, S. Takada, Biophys. J. 85 (2003) 3521. ISSN 00063495.
13. C. M. Dobson, A. Sali, and M. Karplus, Angewandte Chemie International Edition 37, 868 (1998).
14. C. Ricci, M. G. Ortore, S. Vilasi, R. Carrotta, M. R. Mangione, D. Bulone, F. Librizzi, F. Spinozzi, G. Burgio, H. Amenitsch, et al., Biophysical chemistry 208, 68 (2016).
15. C. A. Siedlecki, B. J. Lestini, K. Kottke-Marchant, S. J. Eppell, D. L. Wilson, R. E. Marchant, Blood 88 (1996) 2939. ISSN 00064971.
16. C. E. Sing, A. Alexander-Katz, Biophys. J. 98 (2010) L35. ISSN 00063495. http://linkinghub.elsevier.com/retrieve/pii/S0006349510001979.
17. C. M. Dobson, A. Sali, M. Karplus, A. Sali, M. Karplus, Angew. Chem. Int. Ed. 37 (1998) 868. ISSN 14337851.
18. D. F. Parsons, D. R. M. Williams, Phys. Rev. E 74 (2006) 041804. ISSN 1539-3755.
19. D. I. Dimitrov, A. Milchev, K. Binder, J. Chem. Phys. 125 (2006) 34905. ISSN 00219606.
20. de Marco A, Deuerling E, Mogk A, Tomoyasu T, Bukau B (2007) Chaperone-based procedure to increase yields of soluble recombinant proteins produced in E. coli. BMC Biotechnol 7:1-9.
21. E. De Santis, V. Minicozzi, O. Prolix, G. Rossi, K. I. Silva, M. J. Lawless, F. Stellato, S. Saxena, and S. Morante, The Journal of Physical Chemistry B 119, 15813 (2015).
22. F. Castello, S. Casares, M. J. Ruedas-Rama, and A. Orte, The Journal of

Physical Chemistry B 119, 8260 (2015).
23. F. Weber, F. Keppel, C. Georgopoulos, M. K. Hayer-Hartl, and F. U. Hartl, Nature

Structural & Molecular Biology 5, 977 (1998).
24. G. G. Tartaglia, S. Pechmann, C. M. Dobson, M. Vendruscolo, Trends Biochem. Sci. 32 (2007) 204. ISSN 09680004.
25. I. B. Bekard, P. Asimakis, J. Bertolini, and D. E. Dunstan, Biopolymers 95, 733 (2011).
26. I. Coluzza, A. De Simone, F. Fraternali, and D. Frenkel, PLoS Comput Biol 4, e1000006 (2008).
27. I. Coluzza, S. M. Van Der Vies, and D. Frenkel, Biophysical journal 90, 3375 (2006).
28. I. V. Neratova, T. Kreer, J.-U. Sommer, Macromolecules 48 (2015) 3756. ISSN 0024-9297.
29. J. Frydman, Ann. Rev. Biochem. 70 (2001) 603. ISSN 0066-4154.
30. J. Jaspe, S. J. Hagen, Biophys. J. 91 (2006) 3415. ISSN 00063495.
31. J. Martin and F.-U. Hartl, Proceedings of the National Academy of Sciences 94, 1107 (1997).
32. J. Martin, Biochemistry 41, 5050 (2002).
33. J. D. Honeycutt, D. Thirumalai, Biopolymers 32 (6) (1992) 695. ISSN 10970282.
34. K. Binder, Eur. Phys. J. E Soft Matter 9 (2002) 293. ISSN 12928941.
35. K. L. Prime and G. M. Whitesides, Science 252, 1164 (1991).
36. M. Schor, J. Vreede, and P. G. Bolhuis, Biophysical journal 103, 1296 (2012).
37. M. Vendruscolo, C. M. Dobson, Faraday Discuss. 143 (2009) 277. ISSN 1359-6640.
38. M. Vendruscolo, E. Paci, C. M. Dobson, M. Karplus, Nature 409 (2001) 641. ISSN 00280836.
39. M. A. I. Mahmood, W. Ali, A. Adnan, S. M. Iqbal, J. Phys. Chem. B 118 (2014) 5799. ISSN 1520-6106.
40. M.-B. Luo, D. A. Tsehay, L.-Z. Sun, J. Chem. Phys. 147 (2017) 034901. ISSN 0021-9606.
41. N. Combe and D. Frenkel, Molecular Physics 105, 375 (2007).
42. P. B. Sigler, Z. Xu, H. S. Rye, S. G. Burston, W. A. Fenton, and A. L. Horwich, Annual review of biochemistry 67, 581 (1998).
43. P. Giannozzi, K. Jansen, G. La Penna, V. Minicozzi, S. Morante, G. Rossi, and F. Stellato, Metallomics 4, 156 (2012).
44. P. Roach, D. Farrar, and C. C. Perry, Journal of the American Chemical Society 127, 8168 (2005).
45. Puetz J, Wurm F M (2019) Recombinant Proteins for Industrial versus Pharmaceutical Purposes: A Review of Process and Pricing. Processes 7(8):476.
46. R. Chelakkot, R. G. Winkler, and G. Gompper, EPL (Europhysics Letters) 91, 14001 (2010).
47. R. M. Jendrejack, D. C. Schwartz, J. J. de Pablo, and M. D. Graham, The Journal of chemical physics 120, 2513 (2004).
48. R. M. Jendrejack, E. T. Dimalanta, D. C. Schwartz, M. D. Graham, and J. J. de Pablo, Physical review letters 91, 038102 (2003).
49. R. M. Jendrejack, J. J. de Pablo, and M. D. Graham, The Journal of chemical physics 116, 7752 (2002).
50. R. C. F. Cheung, J. H. Wong, T. B. Ng, Appl. Microbiol. Biotechnol. 96 (2012) 1411. ISSN 0175-7598.
51. S. Alexander, J. Phys. 38 (1977) 983. http://jphys.journaldephysique.org/articles/jphys/pdf/1977/08/jphys_1977_38_8_983_0.pdf%5Cn.
52. S. Egorov, A. Milchev, L. Klushin, K. Binder, Soft Matter 7 (2011) 5669. ISSN 1744683X 17446848.
53. S. Falke, F. Tama, C. L. Brooks, E. P. Gogol, and M. T. Fisher, Journal of molecular biology 348, 219 (2005).
54. S. G. Burston, J. S. Weissman, G. W. Farr, W. A. Fenton, and A. L. Norwich (1996).
55. S. Gräslund, P. Nordlund, J. Weigelt, J. Bray, O. Gileadi, S. Knapp, U. Oppermann, C. Arrowsmith, R. Hui, J. Ming, et al. Nat. Methods 5 (2008) 135. ISSN 1548-7091.
56. S. Plimpton, J. Comput. Phys. 117 (1995) 1. ISSN 0021-9991.
57. S. Schneider, S. Nuschele, A. Wixforth, C. Gorzelanny, A. Alexander-Katz, R. Netz, and M. Schneider, Proceedings of the National Academy of Sciences 104, 7899 (2007).
58. T. X. Hoang, L. Marsella, A. Trovato, F. Seno, J. R. Banavar, and A. Maritan, Proceedings of the National Academy of Sciences 103, 6883 (2006).
59. T. Z. Yuan, C. F. Ormonde, S. T. Kudlacek, S. Kunche, J. N. Smith, W. A. Brown, K. M. Pugliese, T. J. Olsen, M. Iftikhar, C. L. Raston, et al., ChemBioChem 16, 393 (2015).
60. T. R. Jahn, S. E. Radford, Arch. Biochem. Biophys. 469 (2008) 100. ISSN 00039861.
61. U. Shimanovich, I. Efimov, T. O. Mason, P. Flagmeier, A. K. Buell, A. Gedanken, S. Linse, K. S. Akerfeldt, C. M. Dobson, D. A. Weitz, et al., ACS nano 9, 43 (2015).
62. V. N. Uversky, in Protein Chaperones and Protection from Neurodegenerative Diseases (John Wiley & Sons, Inc., 2011), pp. 1-63, ISBN 9781118063903.
63. Y. Rabin, S. Alexander, Europhys. Lett. (EPL) 13 (1990) 49. ISSN 0295-5075.
64. Z. Posel, M. Svoboda, C. M. Colina, M. Lisal, Soft Matter 13 (2017) 1634.
65. Z. Zhou, J.-B. Fan, H.-L. Zhu, F. Shewmaker, X. Yan, X. Chen, J. Chen, G.-F. Xiao, L. Guo, and Y. Liang, Journal of Biological Chemistry 284, 30148 (2009).

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A device for capturing and unfolding a polymeric species or disrupting aggregates of a polymeric species, the device comprising:
(a) a thin support; and
(b) a plurality of nanopore structures piercing through the support, each nanopore structure having an inner surface and a void running the length of the structure, an outer boundary of the void being defined by the inner surface of the nanopore structure,
the inner surface comprising hydrophobic regions capable of capturing and facilitating the unfolding of the misfolded polymeric species.

2. The device of claim 1, wherein the support is of about 10 μm in thickness and about 1 cm² in area.

3. The device of claim 1, wherein the support is made of a material to which the polymeric species do not stick.

4. The device of claim 1, wherein the support is made of silica or aluminum-oxide.

5. The device of claim 1, wherein the nanopore structure has overall cylindrical shape with a diameter ranging from 100 nm to 200 nm.

6. The device of claim 1, wherein the polymeric species is a misfolded protein.

7. The device of claim 1, wherein the polymeric species is passed through the nanopore structures by a pressure driven flow of about 0.004 g to about 0.01 g.

8. The device of claim 1, wherein the inner surface of the nanopore structure has a hydrophobicity (ε_{w}) greater than or equal to 5.0 k_{B}T.

9. The device of claim 1, wherein the hydrophobic regions comprise a plurality of polymer brushes.

10. The device of claim 9, wherein the polymer brushes are made of polymers that is soluble in water.

11. The device of claim 9, wherein the polymer brushes are made of polymers selected from PEG (Polyethylene glycol), PNIPAM (Poly(N-isopropylacrylamide)), or combinations thereof.

12. The device of claim 9, wherein the polymer brushes have a chain length of 10 to 24 monomers.

13. The device of claim 9, wherein the polymer brushes are capable of contacting the polymeric species, said contacting resulting in the disruption of the aggregates of the polymeric species or the unfolding of the polymeric species.

14. The device of claim 9, wherein a flow force is applied across the polymer brushes and creates a density gap at the center of the nanopore structure, and wherein the polymeric species is unfolded if the density gap is smaller than the size of the polymeric species.

15. The device of claim 13, wherein the density gap is between about 2 and about 6 amino acid residues wide.

16. The device of claim 1, wherein the nanopore structure has a radius of about 6 to about 20 amino acid residues.

17. A method of separating an aggregate of polymeric species comprising the steps of:
(a) contacting a solution comprising the aggregate with one side of a device according to claim 1; and
(b) translocating the aggregate of the polymeric species through the nanopore structures of the device by applying a fluid force on the solution.

18. The method of claim 17, wherein the aggregate is a protein aggregate.

19. The method of claim 17, further comprising the steps of:
(c) once all the solution is on the other side of the device, repeating step (b) by applying a fluid force from the opposite direction;
(d) repeating steps (b)-(c) as necessary; and
(e) collecting the solution.

20. The method of claim 17, wherein the device is replaced with a plurality of same devices arranged in series.

21. A method of unfolding a misfolded polymeric species comprising the steps of:
(a) contacting a solution comprising the misfolded polymeric species with one side of a device according claim 1; and
(b) translocating the misfolded polymeric species through the nanopore structures of the device by applying a fluid force on the solution.

22. The method of claim 21, wherein the misfolded polymeric species is a misfolded protein.

23. The method of claim 21, further comprising the steps of:
(c) once all the solution is on the other side of the device, repeating step (b) by applying a fluid force from the opposite direction;
(d) repeating steps (b)-(c) as necessary; and
(e) collecting the solution.

24. The method of claim 21, wherein the device is replaced with a plurality of same devices arranged in series.

25. The method of claim 22, further comprising the step of: allowing the unfolded protein refold into its native conformation.

26. A method of separating a misfolded polymeric species from a mixture of correctly folded native species and misfolded species, the method comprising the steps of:
(a) contacting the mixture with one side of a device according to claim 1;
(b) applying a fluid force on the mixture sufficient to translocate the correctly folded native polymeric species through the nanopore structures of the device while the misfolded polymeric species become associated with the inner surface of the nanopore structures; and
(c) collecting the properly folded polymeric species on the other side of the device.

27. The method of claim 26, wherein the misfolded polymeric species is a misfolded protein.

28. A method of treating a subject suffering from a disease associated with aggregated protein molecules comprising the steps of:
(a) obtaining sufficient amount of a body fluid comprising aggregated protein molecules from the subject;
(b) contacting the body fluid with one side of a device according to claim 1;
(c) passing the body fluid through the nanopore structures of the device by applying a fluid force on the body fluid to disrupt the aggregated protein molecules;
(d) collecting the body fluid on the other side of the device;
(e) repeating steps (b)-(d) as necessary; and
(f) reintroducing the body fluid collected in step (e) into the subject so as thereby to treat the subject.

29. The method of claim 28, wherein the subject is a human.

30. A method of treating a subject suffering from a disease associated with misfolded protein molecules comprising the steps of:
(a) obtaining sufficient amount of a body fluid comprising misfolded protein molecules from the subject;
(b) contacting the body fluid with one side of a device according to claim 1;
(c) passing the body fluid through the nanopore structures of the device by applying a fluid force on the body fluid to unfold the misfolded protein molecules;
(d) collecting the body fluid on the other side of the device;
(e) repeating steps (b)-(d) as necessary;
(f) allowing the unfolded protein molecules in the body fluid collected in step (e) to refold into the native conformation; and
(g) reintroducing the body fluid from step (f) into the subject so as thereby to treat the subject.

31. The method of claim 30, wherein the subject is a human.

32. A system for measuring biomolecule transport, disaggregation and refolding in a liquid sample, comprising: software programmed to run the system, and hardware that controls flow and pressure independently,
wherein the hardware comprises the following devices connected in the following order:
(a) a compressor that generates a pressure;
(b) a pressure controller that controls the pressure generated by the compressor;
(c) a filter;
(d) a reservoir that holds the liquid sample;
(e) a bubble trap and degasser;
(f) a flow sensor that measures the flow rate of the sample;
(g) an extruder in which a membrane with nanochannels is mounted;
(h) a refractive index and/or fluorescence detector to analyze the liquid sample that flows through the membrane; and optionally
(i) an automated collection unit to collect aliquots of the sample.

33. The system of claim 32, wherein the membrane is silicon nitride membrane or anodized alumina membrane.

34. The system of claim 32, wherein the nanochannels have a length ranging from about 300 nm to about 100 μm, and have tunable apertures.

35. The system of claim 32, wherein the membrane with nanochannels is modified with dense polymer brushes.

36. The system of claim 35, wherein the polymer is poly(N-isopropyl acrylamide) (PNIPAM).
